(19) United States
(12) Patent Application Publication
(10) Pub. No.: US 2026/0038684 A1
(43) Pub. Date: Feb. 5, 2026

(54) SYSTEM AND METHOD FOR MONITORING, EVALUATION, AND REPORTING OF LIFE ACTIVITIES

(71) Applicant: EmPowerYu, Inc., Santa Clara, CA (US)

(72) Inventor: Laura J. McIntosh, Santa Clara, CA (US)

(73) Assignee: EmPowerYu, Inc., Santa Clara, CA (US)

(21) Appl. No.: 19/284,198

(22) Filed: Jul. 29, 2025

Related U.S. Application Data
(60) Provisional application No. 63/677,027, filed on Jul. 30, 2024.

(51) Int. Cl. G16H 40/67; G16H 15/00; G16H 50/70

(52) U.S. Cl. CPC G16H 40/67; G16H 15/00; G16H 50/70

(57) ABSTRACT

A system for generating a symptom cluster chart for an individual's condition includes a data analytics tool that creates a multi-axis chart, each axis representing a different condition parameter. It collects data from various sources over a set time, determines the normal range for each parameter, and plots this as a baseline polygon. It also sets acceptable deviation thresholds, forming a second polygon. New data points are plotted and connected to form an updated shape, which is displayed on a user interface to visually track deviations from normal health parameters.

### RELATED APPLICATION

This Application is related to and claims priority to U.S. Provisional Patent Application Ser. No. 63/677,027 filed on Jul. 30, 2024, and entitled “SYSTEM AND METHOD FOR MONITORING, EVALUATION, AND REPORTING OF LIFE ACTIVITIES IN AN UNATTENDED SETTING,” the content of which are incorporated in their entirety herein by reference.

### BACKGROUND

An individual's home is typically a place of comfort and familiarity, where the individual can be themselves and live independently. Therefore, it is not surprising that an increasing number of individuals choose to remain in their homes for as long as possible, despite increasing infirmities as they get older. Increasingly, to keep people in their homes, healthcare is being integrated between the home and clinical settings in order to provide holistic care that improves outcomes. However, an individual's healthcare needs and ability to live independently in a community home can be complex and confusing for the individual, another responsible family member, and to those who care for that individual.

Certain individuals, such as the elderly, those with physical or mental challenges or disabilities, or those who may need monitoring for other reasons, can be particularly vulnerable to issues such as falls, medical events, chronic conditions, environmental triggers (transient, seasonal, or repeating), or sudden illness, which can adversely impact their ability to remain safe in their homes. Naturally, families tend to worry about the safety of loved ones alone in their homes. Also, healthcare organizations want to understand the health status of their patients at home to provide both preventative and proactive care that improves health outcomes and reduces care costs. Such an understanding can often be multifactorial.

Previous attempts to address these issues have often been unsatisfactory for both healthcare providers and the families as they worry about the safety of older and/or disabled adults at home. For example, some families and healthcare systems employ a professional caregiver, such as a homecare nurse or other suitable professional caregiver, to take care of people in their homes. However, use of a professional caregiver tends to be expensive and is only reassuring for the time the caregiver is at the home. In other situations, use of a professional caregiver may be less effective due to issues of availability, as well as potential geographical or language barriers.

Communication technologies, such as phones, emergency buttons and similar devices, have also been used to monitor people in their homes. Unfortunately, such communication technologies tend to be effective only as long as the individual is willing and able to respond to communications into the home or to reach out themselves for help.

Wearable sensors and/or personal emergency buttons can also be used to monitor people in their homes. However, wearable sensors and personal emergency buttons are effective only to the extent the individuals actively cooperate by keeping them charged and actually carrying them at all times. Many elders are either unable to consistently remember, or are not entirely willing to maintain and carry such personal sensors. Moreover, systems using such sensors can often entail excessive volumes of unfiltered, raw, detailed information which may thus be of limited practical use to a monitoring user (such as a family member or a healthcare provider). Too much information similarly tends to create privacy and acceptability issues, for example by showing an individual's exact location in the house or reporting their bathroom or other intimate personal habits. Too much information also tends to create information overload for the monitoring user, requiring the monitoring user to sift through excessive data about normalcy without extracting relevant data about what conduct the subject person is actually engaged in or what conditions the subject person may be suffering from.

Recording technologies, like video cameras, analysis of wave forms like WiFi, and audio surveillance devices, can also monitor people in their homes. However, such technologies are often seen as very intrusive by many individuals, and can be unacceptable to one or another party involved in the monitoring process due to privacy issues.

Thus, in situations in which an individual lives independently but potentially struggles with physical or mental limitations, or chronic medical conditions, and is not interested in wearables, cannot remember how to use a smartphone, or will not allow cameras in their home, family and clinical care providers (i.e. the monitoring users) may prefer to utilize a remote monitoring system or method. As described, it is appreciated that reasons for remote monitoring can include physical, cognitive, economic, geographic, infrastructure, and language barriers to in-person visits.

Unfortunately, even previous systems that incorporate remote monitoring have not been completely satisfactory. Such previous remote monitoring systems have typically focused, in general, on the direct measurement of events or occurrences of interest, on solely physiological measurements, and/or on single factors (of the many factors which may be present or interacting). For example, the focus may be placed on when a monitored individual is in bed, sitting on a particular chair, or opening their medicine chest. Though some pattern detection has been considered, these systems are highly limited in their ability to infer behaviors or events that are not actually measured directly. Generally, piecemeal solutions have not been effective in changing outcomes for the monitored person. This includes medical device measurements, which although they may be relevant to a clinical condition, do not address how well a person is feeling or an ability to adequately self-care in the home. A holistic view of the person is needed for effectively improving outcomes.

While electronic medical records are now typically available to most individuals through patient portals, the electronic medical records again typically focus on individual measurements and factors, and do not provide a means to combine factors in a meaningful way. Only through detailed further analysis of the medical records can the true stability or change over time in an individual's activities and conditions be evaluated. Merely looking at the medical records cannot provide such an evaluation to the individual or the monitoring users.

There is therefore a need for a system and method which provide for effective yet discreet, minimally intrusive monitoring in the home and from personally acceptable digital technologies, integration with clinical data if desired and authorized, and review of an individual's multifactorial components of well-being that enables both direct and indirect measurement and then visualization, recognition, comprehension, and assessment of the stability or change over time in an individual's activities and conditions. There is also a need for such a system and method to offer a simple, manageable, clear and unambiguous presentation of such information to the monitoring user(s), especially when abnormal or unusual conduct is detected.

### SUMMARY

The present invention is directed toward a system for generating a symptom cluster chart relative to at least one condition of an individual in a setting. In various embodiments, the system includes a data analytics resource that is configured to (i) generate three or more input axes for the symptom cluster chart that extend outwardly from a common origin, each of the three or more input axes relating to a condition parameter of the at least one condition; (ii) receive data from a plurality of data sources for each condition parameter relative to a predetermined period of time; (iii) determine a normal range for each condition parameter based on the received data; (iv) assign numeric value along each of the three or more input axes for each condition parameter so that the normal range for each condition parameter forms a first regular polygon shape when plotted and connected along each of the input axes on the symptom cluster chart; (v) establish a threshold of acceptable deviation from the normal range for each condition parameter so that the threshold of acceptable variation for each condition parameter forms a second regular polygon shape when plotted and connected along each of the input axes on the symptom cluster chart; (vi) receive at least one additional data point for each condition parameter; (vii) plot the at least one additional data point for each condition parameter along the three or more input axes on the symptom cluster chart; (viii) connect the at least one additional data point for each condition parameter on the symptom cluster chart to form an updated polygon; and (ix) graphically illustrate the symptom cluster chart on a graphical user interface.

In some embodiments, the second regular polygon shape is larger than the first regular polygon shape.

In many embodiments, at least one of the plurality of data sources is positioned within the setting.

In several embodiments, at least one of the plurality of data sources is positioned outside the setting.

In certain embodiments, the at least three input axes are weighted relative to one another within the symptom cluster chart.

In some embodiments, if the at least one additional data point from any of the plurality of data sources is outside the threshold of acceptable deviation for the condition parameter, then the symptom cluster chart graphically illustrated on the graphical user interface changes in one or more of shape, size, fill pattern, and color.

In certain embodiments, if each additional data point from the plurality of data sources is outside the threshold of acceptable deviation for the condition parameter, then the symptom cluster chart graphically illustrated on the graphical user interface changes in both size and color.

In various embodiments, the condition parameters relevant to the at least one condition of the individual include one or more sleep activities, eating activities, movement activities, environmental readings, and medical device readings relative to the individual.

In certain embodiments, the condition parameters relevant to the at least one condition of the individual include the sleep activities of the individual; and the sleep activities of the individual include sleep time, wake time, sleep duration, and sleep gaps as sensed or inferred by the plurality of data sources.

In some implementations, the at least one condition of the individual includes frailty; and the condition parameters include (i) eating/weight, (ii) amount/distribution of activity, (iii) walking speed, (iv) fatigue, and (v) socialization.

In other implementations, the at least one condition of the individual includes congestive heart failure; and the condition parameters include (i) pillow count, (ii) blood pressure, (iii) activity at night, (iv) fatigue, (v) amount of movement during day, (vi) weight, (vii) sleep gaps, and (viii) oxygen saturation.

In many embodiments, the system further includes a data collection resource that is configured to collect and store the data from the plurality of data sources for each of the condition parameters.

In certain embodiments, the data collection resource is a cloud-based system.

In some embodiments, the data analytics resource is incorporated within the data collection resource.

The present invention is further directed toward a method for generating a symptom cluster chart relevant to at least one condition of an individual in a setting, including steps of (i) generating with a data analytics resource three or more input axes for the symptom cluster chart that extend outwardly from a common origin, each of the three or more input axes relating to a condition parameter of the at least one condition; (ii) receiving data from a plurality of data sources for each condition parameter relative to a predetermined period of time with the data analytics resource; (iii) determining a normal range for each condition parameter based on the received data with the data analytics resource; (iv) assigning numeric value along each of the three or more input axes for each condition parameter with the data analytics resource so that the normal range for each condition parameter forms a first regular polygon shape when plotted and connected along each of the input axes on the symptom cluster chart; (v) establishing a threshold of acceptable deviation from the normal range for each condition parameter with the data analytics resource so that the threshold of acceptable deviation for each condition parameter forms a second regular polygon shape when plotted and connected along each of the input axes on the symptom cluster chart; (vi) receiving at least one additional data point for each condition parameter with the data analytics resource; (vii) plotting the at least one additional data point for each condition parameter along the three or more input axes on the symptom cluster chart with the data analytics resource; (viii) connecting the at least one additional data point for each condition parameter on the symptom cluster chart with the data analytics resource to form an updated polygon; and (ix) graphically illustrating the symptom cluster chart on a graphical user interface with the data analytics resource.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic illustration of an embodiment of a monitoring system having features of the present invention that is utilized to monitor, evaluate and report life activities and conditions of an individual in an unattended setting;

FIG. 2 is a representative example of a tabular illustration of sleeping activities, eating activities, movement activities, and medical device readings for the individual, and temperature readings for the unattended setting, which can be incorporated within functions of the monitoring system;

FIG. 3 is a simplified graphical illustration showing the general process for use of the monitoring system, including analyzing symptoms clustered together to assess specific syndromes;

FIG. 4 is a simplified graphical illustration showing a visualized compilation data set, in the form of a symptom cluster chart, which can be used within the monitoring system to provide information about the individual;

FIG. 5A is a simplified graphical illustration of factors usable within the symptom cluster chart when reviewing potential frailty of the individual with the monitoring system;

FIG. 5B is a simplified graphical illustration showing normal values and threshold values for each of the factors usable within the symptom cluster chart when reviewing potential frailty of the individual with the monitoring system;

FIG. 5C is a simplified graphical illustration showing actual values for each of the factors usable within the symptom cluster chart when reviewing potential frailty of the individual with the monitoring system;

FIG. 5D is another simplified graphical illustration showing actual values for each of the factors usable within the symptom cluster chart when reviewing potential frailty of the individual with the monitoring system;

FIG. 6 is a simplified graphical illustration of factors usable within the symptom cluster chart when reviewing potential congestive heart failure of the individual with the monitoring system;

FIG. 7A is a simplified illustration and description of how the symptom cluster chart tracks progression of actual values for each of the factors included when reviewing any potential condition of the individual with the monitoring system;

FIG. 7B is another simplified illustration and description of how the symptom cluster chart tracks progression of actual values for each of the factors included when reviewing any potential condition of the individual with the monitoring system;

FIG. 7C is still another simplified illustration and description of how the symptom cluster chart tracks progression of actual values for each of the factors included when reviewing any potential condition of the individual with the monitoring system;

FIG. 7D is a simplified illustration and description of how one of the factors included within the symptom cluster chart can be further broken down into a plurality of factor inputs when reviewing a potential condition of the individual with the monitoring system;

FIG. 8 is a simplified flowchart demonstrating a system and method for generating a symptom cluster chart having features of the present invention;

FIG. 9 is a representative example of a healthcare data visualization dashboard that can be illustrated within a graphical user interface, including an example of the symptom cluster chart with time progression as shown in FIG. 7B;

FIG. 10A is a simplified illustration of an example top level view of a general review of the individual regarding one or more activities and environmental readings relevant to the individual that can be provided through use of the monitoring system;

FIG. 10B is a simplified illustration of an example second level, summary view of a general review of the individual regarding one or more activities and environmental readings relevant to the individual that can be provided through use of the monitoring system;

FIG. 11 is a series of related graphical illustrations usable to review sleep activities of the individual that can be provided through use of the monitoring system;

FIG. 12 is a series of related graphical illustrations usable to review eating activities of the individual that can be provided through use of the monitoring system;

FIG. 13 is a series of related graphical illustrations usable to review movement activities of the individual that can be provided through use of the monitoring system;

FIG. 14 is a series of related graphical illustrations that are usable to track medical device usage of the individual that can be provided through use of the monitoring system;

FIG. 15 is a series of related graphical illustrations usable to review sleep, meal preparation, and movement activities of the individual that can be provided through use of the monitoring system, which may differ from what has been verbally reported by an individual having issues of dementia;

FIG. 16A is a simplified illustration of an example top level view of a general presentation regarding a potential condition of the individual that can be provided through use of the monitoring system; and

FIG. 16B is a simplified illustration of an example detailed view of certain factors for the general presentation regarding the potential condition of the individual of FIG. 15A that can be provided through use of the monitoring system.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

In various embodiments, the present invention is directed toward a system and corresponding method for monitoring, visualizing, describing, quantifying, and reporting experienced daily life activities and conditions of an individual in a setting. In such embodiments, the daily life activities and conditions of the individual in the setting can be visualized, reported and/or demonstrated through generation of a symptom cluster chart that illustrates normal, baseline life activities and conditions of the individual, and then further illustrates any variations from normal based on current data. As described in detail herein, the symptom cluster chart includes three or more input axes that are each representative of condition parameters or variables relative to the experienced daily life activities and conditions of the individual, and that incorporate (1) discrete data received from each of a plurality of data sources, and/or (2) derived information that is inferred through multi-factorial combinations of data received from at least two of the plurality of data sources. The symptom cluster chart can be made available and be viewed by any monitoring users that are monitoring the daily life activities and conditions of the individual in the setting. As further described herein, the symptom cluster chart is provided in such a manner that any variations of the daily life activities and conditions of the individual away from normal are presented in a manner that is simple, manageable, clear and unambiguous.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It is appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is recognized that such a development effort might be complex and time-consuming, but would nevertheless be a routine undertaking of engineering for those of ordinary skill in the art having the benefit of this disclosure.

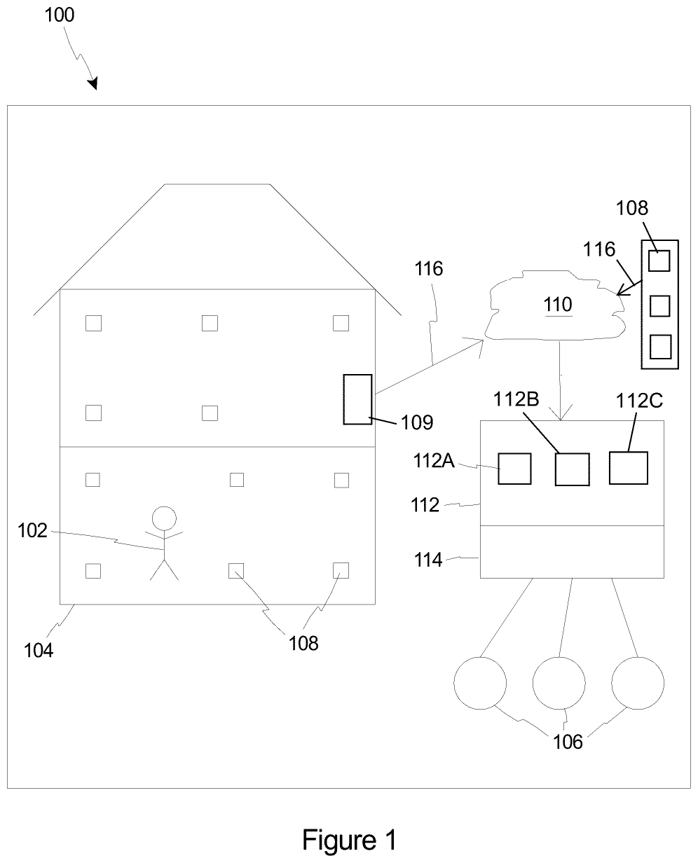

FIG. 1 is a simplified schematic illustration of an embodiment of a monitoring system 100, such as a remote monitoring system in certain non-exclusive embodiments, having features of the present invention that is utilized to monitor, evaluate and report life activities and conditions of an individual 102 in an unattended setting 104 (also sometimes referred to as a “setting”), such as a home of the individual 102. In some applications, the individual 102 can be an elderly person who can be of high medical risk, but who does not use digital health technology. In other applications, the individual 102 can be someone who may not necessarily be of advanced age, but who may have certain physical or mental disabilities. In still other applications, the individual 102 may be someone who may be able to function independently in many degrees, but who for various other reasons may have limited ability to call for help or report problems during unusual or unexpected situations. In yet other applications, the individual 102 may be any other type of individual who others believe needs to be monitored due to an inability to reliably and responsibly protect themselves when in an unattended setting 104. One or more monitoring users 106, such as family members, loved ones, caregivers, clinical staff, or designated responders, that utilize the monitoring system 100 to monitor the individual 102 in the unattended setting 104 are also shown in FIG. 1.

While it is appreciated that the present application generally refers to usage of the monitoring system 100 in an unattended setting, i.e. without others present within the setting 104, it is further appreciated that the monitoring system 100 can be used in any setting 104, including those where others (including any monitoring users 106) other than the individual 102 are present.

The design and specific features of the monitoring system 100 can be varied. In various embodiments, the monitoring system 100 can include one or more of (i) a plurality of data sources 108 (illustrated as a series of small boxes), such as sensors, monitors, medical devices, measurement devices, medical records, or other suitable data sources, that can be positioned in disparate locations throughout the setting 102 and/or can be positioned outside the setting 102, (ii) an encryptor 109, which can be used to selectively encrypt any data collected from or by the plurality of data sources 108, such as for privacy purposes, (iii) a data collection resource 110 that is configured to receive and/or collect data from the plurality of data sources 108, (iv) a data analytics resource 112 that can include any suitable software, hardware, processors, algorithms, etc. for purposes of utilizing, analyzing, interpreting, combining and generating useful output from the data from the plurality of data sources 108, and (v) a graphical user interface 114 (or “GUI”) for visually displaying the output generated by the data analytics resource 112, such as in the form of a symptom cluster chart. In many embodiments, the data collection resource 110 can be a cloud-based system like a cloud connected server, and the data analytics resource 112 can be incorporated within the data collection resource 110. Alternatively, the data analytics resource 112 can be provided independently from the data collection resource 110. Still alternatively, the monitoring system 100 can have more components or fewer components than those specifically noted herein. For example, in certain embodiments, the monitoring system 100 can be designed without the specific use of the encryptor 109.

As an overview, as described in detail herein, the monitoring system 100 serves to provide direct and/or indirect, event-based monitoring of the individual 102 within the setting 104 to identify anomalous conduct indicating potential adverse consequences for the health and well-being of the individual 102. Depending on the particular application, the monitoring system 100 can be used to determine patterns of activity through event sensing and monitoring with sufficient efficacy, for instance, to discriminate those situations where a lack of sensed activity is due to absence of the monitored individual 102 within the setting 104 from other situations where it is actually due to a lack of activity by the monitored individual 102 though present. In certain applications, the monitoring system 100 employs indirect measures computed from information acquired through available data sources 108 to determine the presence, absence, and/or degree of one or more target activity types or condition parameters. In certain applications, the monitoring system 100 generates a time-cycle, activity portrait model of the individual 102, and, based on such model, measures the degrees to which new events are consistent or anomalous. The data analytics resource 112 can thus review the status and condition of the individual 102 based on the sensed activities and/or other available data relating to the individual 102, which can then be reported to the monitoring users 106 in a clear and unambiguous manner through use of the GUI 114, such as by employing visualized compilation data sets which may be in the form of symptom cluster charts in various embodiments. It is appreciated that the monitoring system 100 of the present invention thus provides the ability to integrate vastly different types of data into one summary graphic relevant to the specific individual 102, as well as to describe and deploy variables that are specific to the situation, but which may only exist anecdotally, such as with weight gain/loss guidelines, typical eating habits, typical sleeping habits, typical movement activities, medical readings, typical social behaviors, etc.

As so described, the monitoring system 100 can be utilized to monitor, visualize, quantify, describe, and report any desired activities and conditions of the individual 102. For example, the individual 102 can have symptoms and/or behavioral manifestations that can be effectively and accurately quantified through use of the monitoring system 100 relevant to physical, mental and/or medical conditions including, but not limited to, frailty, congestive heart failure, sundowning (which generally refers to the increase in activity, agitation, confusion, or delirium seen at the end of the daylight period in persons with disturbed circadian rhythms, and which is common in persons diagnosed with many stages and types of dementia, especially Alzheimer's Disease), dementia and other memory-related conditions, urinary tract infection, kidney disease or failure, respiratory conditions like asthma, pneumonia, and chronic obstructive pulmonary disease, disabling falls or stroke, depression, pain from conditions like arthritis or sickle cell disease, cancer treatment side effects, chronic obstructive lung disease, etc. It is appreciated that the behavioral manifestations can also result from multiple concurrent conditions.

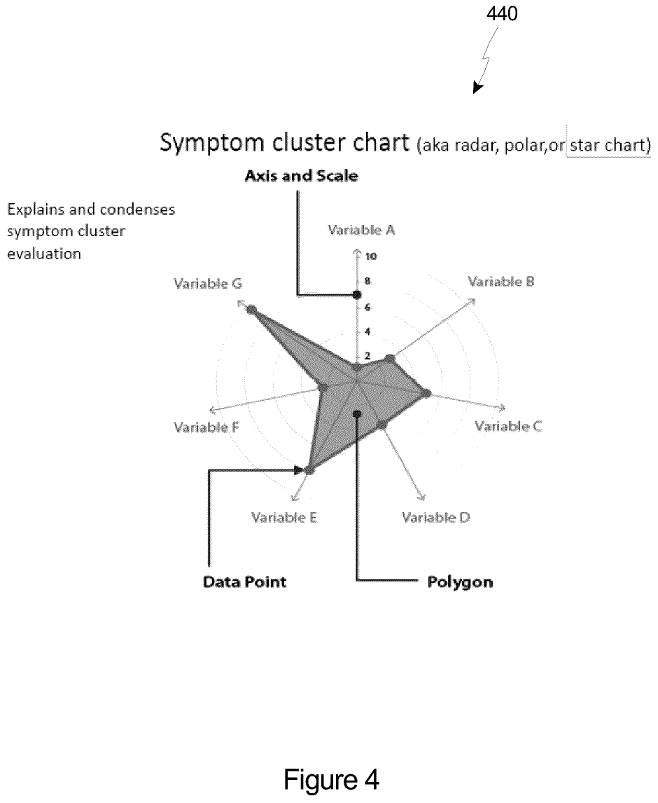

The plurality of data sources 108, which, as noted, can be positioned in disparate locations throughout the setting 104, and/or external to the setting 104, can be of any suitable types, and can be used to monitor (directly or indirectly) any activities or measurements of the individual 102 that are deemed proper and/or necessary to monitor. In particular, the data sources 108 can be utilized to monitor and/or sense any activities that occur within the setting 104, and which can then generate data 116 that can be used and/or analyzed within the data analytics resource 112 to effectively and accurately detect changes and understand status regarding the various potential physical, mental and/or medical conditions of the individual 102. More specifically, in various embodiments, each of the plurality of data sources 108 is configured to provide data 116 that is usable, directly or in combination with data 116 from other data sources 108, to describe a condition parameter relative to the activities and/or conditions of the individual 102 within the setting 104. Additionally, each of the condition parameters is usable to generate a corresponding data point that can be plotted within a visualized compilation data set such as a symptom cluster chart 440 (illustrated, for example, in FIG. 4).

The condition parameters used and/or recognized within any analyzed condition can be varied. For example, in certain non-exclusive applications, the condition parameters can include one or more of eating activities (timing, duration, amount (calories, preparation time), consistency, etc.), weight measurement, movement activity (amount and/or distribution, timing, consistency of pattern), walking speed, socialization evaluation, sleeping activities (including timing, duration, sleep gaps, pillow usage, etc.), blood pressure measurement, pulse measurement, daytime activity vs. nighttime activity, fatigue, oxygen saturation, bathroom usage (including subdivisions such as toilet and shower/tub), location measurements (including timing, duration, activity therein, etc.), specific appliance or device usage (including timing, duration, consistency, etc.), environmental conditions (such as temperature, humidity, light level, barometric pressure, indoor/outdoor air quality, noise, etc.), etc. Additionally, and/or alternatively, the sensed condition parameters can include other suitable sensed condition parameters depending upon the particular condition to be evaluated. Certain non-exclusive specific examples for effectively employing one or more of the noted condition parameters include barometric pressure being a known trigger for arthritis pain, which could affect walking speed, and which can further be a measure of frailty; and humidity in the bathroom being an indicator of personal hygiene, which could further be an indicator of degree of fatigue or dementia.

The encryptor 109 can be utilized to encrypt certain data from the data sources 108, such as for privacy purposes, before such data 116 is transmitted to the data collection resource 10. More particularly, in certain embodiments, data from one or more of the plurality of data sources 108 can be sent to the encryptor 109, which can then receive and encrypt the data 116 before the data 116 is transmitted to the data collection resource 110. Additionally, and/or alternatively, data 116 from the data sources 108 can be transmitted to the data collection resource 110 without first being sent to the encryptor 109 to be encrypted.

As shown in FIG. 1, data 116 collected from the data sources 108, whether or not the data 116 has been encrypted by the encryptor 109, can be transmitted to the data collection resource 110, such as a cloud-based system like a cloud connected server, where the data 116 can be stored. As further shown, the data collection resource 110 can also function as a gateway through which the data 116 is transmitted to the data analytics resource 112. In many embodiments, as noted above, the data analytics resource 112 can be incorporated within the data collection resource 110, such that the data 116 received within the data collection resource 110 will further be analyzed and interpreted within the data analytics resource 112 without the need for transmitting the data 116 externally from the data collection resource 110 to the data analytics resource 112.

As further noted above, the data analytics resource 112 can include any suitable software 112A, hardware 112B, processors 112C, algorithms, etc. for purposes of utilizing, analyzing, interpreting, combining and generating useful output from the data 116 received from the plurality of data sources 108. As described in greater detail herein below, the data analytics resource 112 can utilize, analyze, interpret, and/or combine the data 116 from the data sources 108 in any suitable manner for purposes of generating useful output such as in the form of a symptom cluster chart 440, derived from three or more condition parameters, which can provide information and status relevant to any physical, mental and/or medical conditions of the individual 102.

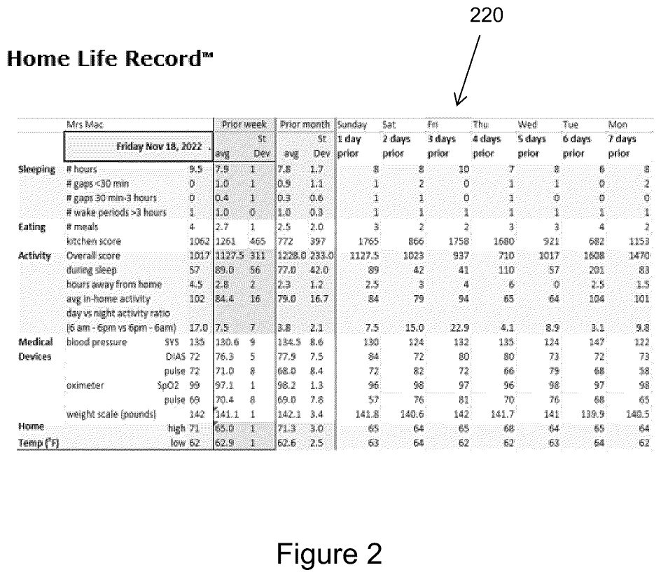

It is appreciated that, in certain instances, the data 116 collected from the data sources 108 can appear misleading, such as with a potential lack of data 116 at certain times, depending on whether or not the individual 102 is actually at the setting 104 at any given time, or when the individual 102 may be sleeping. As such, the monitoring system 100 can act as a state machine to determine a “home state” of the individual 102, which refers to the status of the individual 102 in relation to the setting 104. Generally speaking, the monitoring system 100 can be used to determine four different home states, (i) active in home (in which movement and/or activity is detected within the setting 104), (ii) inactive in home (in which little or no movement and/or activity is detected within the setting 104, but without evidence that the individual 102 has left the setting 104 or that the individual 102 is likely to be sleeping (based on typical sleeping times and locations, or direct data from a suitable data source 108)), (iii) sleeping, and (iv) away from home (or “out of house”). As described, the data 116 generated from the data sources 108 will be interpreted in a different manner depending on the appropriately identified home state of the individual 102 at any given time. For example, the determined home state of “away from home” or “out of house” used in the algorithm requires activity in an exit sensor, or use of a proximity sensor, followed by a lack of activity in the setting 104. When the “out of house” state is so determined, the lack of activity within the setting 104 is not seen as a concern, whereas a lack of activity in the setting 104 when the individual 102 is there may be worthy of concern and/or follow-up. For example, as seen in FIG. 2, hours away from home can be a metric for a Home Life Record® 220 (illustrated in FIG. 2), and are additionally a component of symptoms related to assessing such condition parameters as fatigue and socialization.

Additionally, if an inactivity at home state is determined, then an “inactivity alert” can be sent or otherwise provided to the monitoring users 106. As utilized herein, the term “inactivity alert” refers to a home state that is set as active at home which then changes to inactive at home. The monitoring system 100 then waits for a pre-set time, for example four hours in one non-exclusive implementation, and if activity is detected within the pre-set time, the home state is changed back to active at home. If no activity is detected within the pre-set time, an inactivity alert is sent to the monitoring users 106. The inactive at home state determination and subsequent inactivity alert is intended to detect conditions such as a disabling fall or stroke. This information could then be passed on to the monitoring users 106 and recorded numerically in the Home Life Record® 220 and displayed visually in the symptom cluster chart 440.

As noted, the plurality of data sources 108 can be of any suitable types, can be positioned at any suitable locations within or outside the setting 104, and can be used to directly and/or indirectly sense and monitor any suitable activities or conditions of the individual 102 within the setting 104. For example, the plurality of data sources 108 can include one or more of (1) motion sensors, which can be used in logic trees for determining sleeping, eating, location duration, placement of home in home states (as noted above), detection of “away from home” state, and various activity measures used in behavioral matrix; (2) motion sensors, which can be used in strategic locations such as bedrooms, hallway, bathroom, kitchen, door entryway, living room, hobby room, etc., with each location feeding specific algorithms usable for detection of activity related to the four possible home states, as well as overall activity scores that may be geared toward specific activities in specific areas of the home (such as sleep activities in the bedroom, eating activities in the kitchen, etc.); (3) indoor environmental and energy management-related sensors such as temperature sensors, which can be used for alerts when a temperature value is outside of an established threshold (such as between 55 degrees F. and 85 degrees F., in one non-exclusive example), with raw data being a factor in behavior factor analyses, humidity sensors, which may be relevant in determinations regarding personal hygiene, thermostats, air quality, etc.; (4) time sensors, which can be used for determining and/or deriving normal (or expected) wakeup times, going to sleep times, time spent in specific locations, time measurements between detected events and inferred activities, etc., for the individual 102 for each day of the week, as well as any potential variations therefrom once the normal (or expected) times have been determined; (5) home security-related sensors such as locks, or door/window separation sensors, which can report when two sides of sensor are separated for purposes of determining when the door/window is opened and closed—it is appreciated that such sensor types can further be used for determining opening and closing of any contained space such as medicine cabinets, drawers, removing the top of a containment vessel such as a pill bottle, moving an item from a desired location, and extracting a unit from a storage receptacle, etc., in addition to being used for any and all doors and windows; (6) smart plugs, which can be used to detect, control, and report on/off status and power level of any household appliances for activities including food preparation or cleaning; (7) smart plugs, which can be used to detect, control, and report on/off status of entertainment appliances like televisions or audio systems, as well as any lights at night throughout the setting 104; (8) smart plugs, which can be used to detect, control, and report on/off status of work space related devices such as computers, printers, etc.; (9) home safety-related sensors such as smoke alarms, water detectors, air quality monitors, which can be used to measure air quality and record and provide alerts when air quality falls outside any established threshold, with raw data being a factor in the behavior factor analyses, or carbon monoxide detectors, which can be used to measure and record carbon monoxide levels and provide alerts when the value is outside and established threshold, such as alerting to possible carbon monoxide poisoning, with raw data being a factor in the behavior factor analyses; (10) measurement or emission devices enabled with home automation wireless protocols, with such devices including light bulbs, thermometers, humidity sensors, barometers, vibration sensors, door locks, smoke alarms, water presence, cameras, etc.; (11) toilet use detectors, which can be used to determine quantity and/or quality of toilet use; (12) data sources for which an application programming interface (API) or other means of data transmission from a unit to the data collection resource 110 is created by a device manufacturer to send data to a client, and are generally internet-enabled; (13) internet-enabled medical devices such as weight scales, blood pressure monitors, pulse monitors, oxygen saturation readers, blood glucose monitors, heart rate monitors, medication minders, etc.; (14) environmental measuring devices such as air quality monitors for particulates and volatile organic compounds (including but not exclusive to carbon monoxide and formaldehyde), thermometers for temperature, sound level meters (including for factors such as decibel level, frequency and pitch, timbre, localization, reverberation, etc.); (15) health and fitness-related sensors or devices, such as scales, glucose monitors, blood pressure monitors, pulse monitors, exercise bikes, etc., for determining patterns of usage and when sensed values are outside an expected range; and (16) noise or audio monitors, which can be used to measure noise or sounds in the local environment and to provide alerts when the sounds are unusual or the value is outside established thresholds, with raw data being a factor in the behavior factor analyses. Additionally, and/or alternatively, the plurality of data sources 108 can include other sensor types not specifically disclosed herein.

The terms “bathroom use” and “toilet use” as utilized herein are intended to detect and quantify presence of the individual 102 in this location. In some applications, the identified location of the individual 102 in the bathroom can be restricted, for example only to the toilet area or vanity area. It is further appreciated that such identified “bathroom use” or “toilet use” can be combined with humidity data to report a personal hygiene measurement, which is some implementations could be an input axis and a condition parameter of a symptom cluster chart 440.

As thus described, the data 116 collected and/or generated through use of the plurality of data sources 108 is transmitted to and received by the data analytics resource 112, typically via the gateway provided by the data collection resource 110, so that the activities and/or conditions of the individual 102 can be effectively monitored and evaluated through the algorithms developed, and the software 112A, the hardware 112B, and the processors 112C incorporated within the data analytics resource 112. More specifically, in many embodiments, the data analytics resource 112 and/or the software 112A, the hardware 112B, the processors 112C, and the algorithms, are configured to receive the data 116 from each of the plurality of data sources 108 over a predetermined period of time to determine an expected parameter state for each of the condition parameters relative to the individual 102 within the setting 104. The data analytics resource 112 is then configured to determine a normal range for the expected parameter state for each of the condition parameters relative to the individual 102 within the setting 104.

It is appreciated that the predetermined period of time for receiving data 116 from the plurality of data sources 108 to determine an expected parameter state for each of the condition parameters can be varied, noting that various sensed data can vary depending on the hour of the day, day of the week, time during the month, as well as incorporating seasonal variability. Accordingly, in many non-exclusive embodiments, the predetermined period of time for receiving data 116 from the plurality of data sources 108 to determine an expected parameter state for each of the condition parameters can be at least approximately one week, two weeks, three weeks, one month, two months, three months, four months, five months, or six months. Alternatively, the predetermined period of time for receiving data 116 from the plurality of data sources 108 to determine an expected parameter state for each of the condition parameters can be greater than approximately six months or less than one week.

The data analytics resource 112 can then assess the data 116 for a threshold of acceptable deviation (versus unacceptable deviation, which could warrant an alert or other notification to the monitoring users 106) from the normal range for the expected parameter state for each of the condition parameters relative to the individual 102 within the setting 104.

It is appreciated that the threshold of acceptable deviation from the normal range for the expected parameter state for each of the condition parameters can be determined in any suitable manner. For example, in certain non-exclusive embodiments, the threshold of acceptable deviation from the normal range for the expected parameter state for each of the condition parameters can be based on a percentage variation either above or below the normal range, a ratio assessment which can extend either above or below the normal range, an absolute value of variation either above or below the normal range, a statistical or algorithmic formula for determining an acceptable deviation from the normal range, or another suitable threshold determination method, including by preference of the observing care giver or by medical convention (for example, defined thresholds for normal and high blood pressure). In some implementations, acceptable deviation or thresholds of concern can be set in the monitoring system 100 with clinical or family care giver input, although such thresholds can be set according to statistical or other means from data 116 collected relevant to the individual 102. For example, averaging daily data for the time the individual 102 gets out of bed for the day during the last four months, and setting a threshold of concern at two standard deviations earlier or later than that average time. As such, the monitoring system 100 is configured to reduce the sensor-provided data 116 to expected parameter states with acceptable deviation therefrom for condition parameters in an automated manner.

It is further appreciated that in some applications of the monitoring system 100, survey data from the individual 102, the monitoring users 106, or others can be used to supplement the data 116 collected from the plurality of data sources 108 for purposes of determining the expected parameter state for each of the condition parameters relative to the individual 102 within the setting 104.

Once the normal range and the established threshold have been determined for the expected parameter state for each of the condition parameters, the data analytics resource 112 is configured to receive at least one additional data point from each of the plurality of data sources 108, and to compare the at least one additional data point from each of the plurality of data sources 108 to the normal range for the expected parameter state for each of the condition parameters to determine if the at least one additional data point from each of the plurality of data sources 108 is outside the threshold of acceptable deviation from the normal range for the expected parameter state for each of the sensed condition parameters relative to the individual 102 within the setting 104. As described in greater detail herein below, the data analytics resource 112 can further be configured to generate a visualized compilation data set, such as a symptom cluster chart 440, that graphically illustrates the comparison of the at least one additional data point from each of the plurality of data sources 108 to the normal range for the expected parameter state for each of the condition parameters relative to the individual 102 within the setting 104. In various embodiments, the GUI 114 can then be utilized to provide a visual display to the monitoring users 106 that shows the visualized compilation data set such as the symptom cluster chart 440 in a manner that is clear and unambiguous, so that the monitoring users 106 can quickly and easily determine if action is required based on the reviewed activities and/or conditions of the individual 102 in the setting 104. Additionally, the monitoring users 106 can set thresholds in the visualized compilation data set for automated alerts based on the evaluated activities and/or conditions of the individual 102 in the setting 104.

During the establishment or determination of normal activity levels and threshold variability therefrom, it is appreciated that suitable software and hardware measures for machine learning known in the art may be employed to carry out such automated decision-making processes. In some embodiments, such software and hardware within the data analytics resource 112 can effectively determine a plurality of predefined pattern event scenarios, and the conditions required for detection as anomalous conduct. These can be recorded in a set of expert system rules and acted upon based on logic described according to the sensor inputs that have been tokenized into events of specific types.

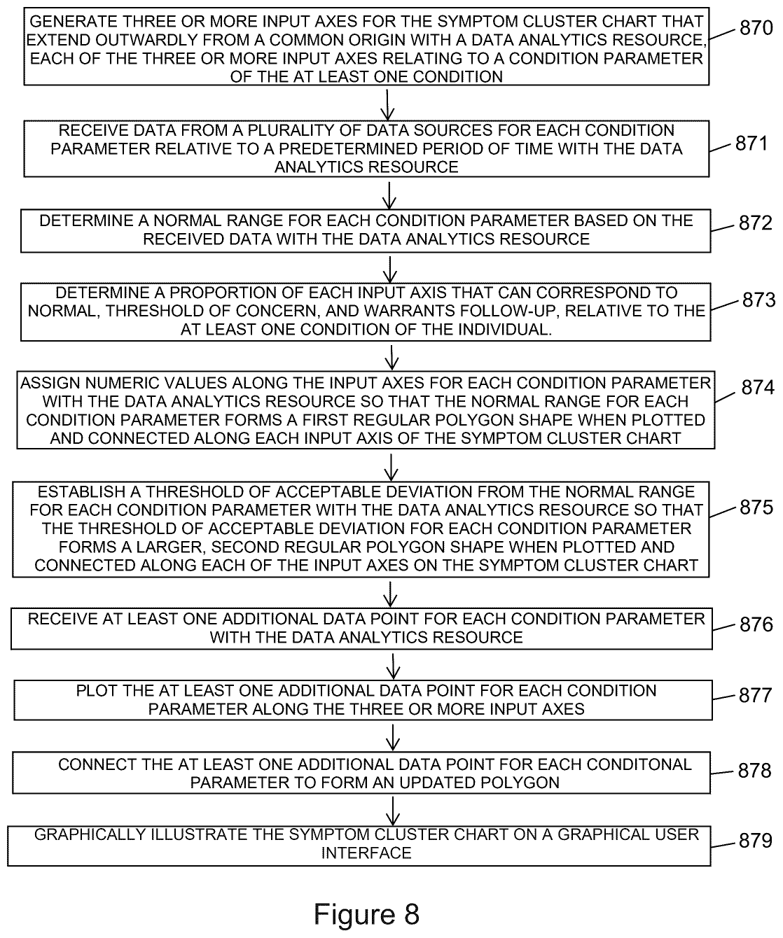

As so described, the monitoring system 100 can be said to include five basic components that illustrate the ability to generate data that will support AI-based behavioral analytics, as well as providing continuous and automated risk assessment. In particular, the monitoring system 100 includes (1) multimodal data sources 108 that are strategically placed throughout and outside the setting 104 to collect data 116 relative to the activities of the individual 102 within the setting 104, and create a labeled dataset that either includes or allows derivation of such labels as frequency, sequence, location, and duration useful for semi-supervised machine learning, (2) a state machine that is utilized to interpret the collected data in context, such as by using software to directly and/or indirectly create information about typical daily activities regarding sleeping activities, eating activities, movement activities, environmental conditions, and medical device usage, (3) an inference engine that uses expert knowledge to interpret and build on sensor data, and be used for machine learning algorithms and techniques, (4) machine learning to create personalized parameters for each individual 102 in each setting 104 (or home), and (5) rich dataset useful for time-series neural network analysis and activity mapping shown to detect abnormal behavior. Based on such basic components, the monitoring users 106 can then receive relevant information through visual presentation on the GUI 114 in the form of a dashboard or numeric table for analytics (such as shown in FIG. 2 and/or FIG. 8), including the use of the visualized compilation data sets.

Upon selective reduction of the processed data to manageable form, the monitoring system 100 delivers the resulting information to the monitoring users 106 and presents the information in a clearly and succinctly summarized graphic display form that may be easily understood and acted upon by even a non-technical monitoring user 106. The monitoring users 106 are thereby alerted via “at-a-glance” status update displays with the GUI 114 in a clear, reliable way when a potential threat to the monitored individual's well-being is determined based on detection of sufficiently anomalous conduct within the setting 104. Use of the monitoring system 100 can thus provide benefits such as early detection of problems, increased staff time efficiency, use of unlicensed care managers for triage of alerts, and timely care that improves the quality of life of the monitored individual 102, which can be done non-intrusively and without requiring the monitored individual 102 to remember any cooperative action or to necessarily do anything to facilitate the data collection.

As so implemented in various embodiments, the monitoring system 100 unobtrusively monitors activity of the individual 102 within the setting 104, and provides reassurances to the monitoring users 106 such as:
1. Notifications re Daily Activities—the monitoring system 100 can generate and deliver suitable notices to the monitoring users 106 when important daily events for the monitored individual 102 occur, such as the first activity of the day, the refrigerator being opened, or the like. Notifications may also be sent out when activities occur which are not within an expected, typical pattern, such as activity occurring during the middle of the night and activity occurring at a time when the individual 102 is expected to be away from the setting 104, among others. Appropriate notification parameters may be selectively set by the monitored individual 102, the monitoring users 106, or by the monitoring system 100 itself according to a default setting. Notifications of the location of the monitored individual 102, including when a monitored individual 102 has dementia or other condition under which the monitoring user 106 restricts access to specified locations or devices, can include an on-site monitoring user 106 choosing to receive an alert in near real time from data sources 108 for locations of concern, such as opening the front door, accessing a chemicals storage space, or turning on an appliance such as a stove that could pose a danger to monitored individual 102.
2. Analytics and Detection of Unusual Events—the monitoring system 100 can create an activity pattern from the acquired sensor data, then alert the monitoring users 106 if an unusual event occurs or an expected activity fails to occur.
3. Activity Log—the monitoring system 100 can establish and maintain a baseline of activity that may be used as a comparative reference, in order to proactively detect for instance upward or downward behavior trends, and/or to scan for anomalous behaviors outside the normal range or beyond established thresholds. The activity log can also follow long-term trends that can be updated over time as new trends may appear, such as due to changes in activities, medications, etc.

As noted herein, some data 116 collected from the plurality of data sources 108 is usable for direct detection of activities and conditions of the individual 102. For example, sensor data 116 can be directly usable to determine ordinary daily activities generally experienced by most human subjects in their normal course, such as sleep and wake times, meal times, TV or media device use times, arrival and departure times, and general patterns of household activity.

However, as further noted herein, other data 116 collected from the plurality of data sources 108 is usable for indirect detection of activities and conditions of the individual 102, such as more detailed analysis of sleeping activities and eating activities of the individual 102. For example, analysis of sleeping activities can be indirectly detected through use of door sensors (for the individual 102 opening and closing bedroom doors and/or bathroom doors), occupancy of household items such as beds, chairs, toilets, carpets, etc. using pressure or vibration sensors (e.g., for sensing weight pressure of the individual 102 when lying in bed), motion sensors (for sensing movement about the bedroom during times in which sleep is expected), power (on/off) sensors (for sensing when lights, TVs, etc. may be turned on or off within the bedroom), light sensors for activities of the individual 102 after sundown such as reading or watching TV when lying in bed and to differentiate from sleep, etc.

As referred to herein, “sleeping activities”, “sleep” or “sleeping” includes tracking of start and wake times, and the number and amount of time of gaps during a total sleep period in which activity is detected. For example, some people get up for an hour in the middle of the night to read and go back to sleep. That would be recorded as a gap between two sleep states, and the two sleep periods would be added together. Such a gap would be categorized differently than a gap of minutes from going to the bathroom for toileting or getting water, then going back to sleep.

Additionally, analysis of eating activities require more than simply detecting when the individual 102 enters or exits the kitchen, and when the refrigerator is opened and closed. More particularly, analysis of eating activities can further include one or more motion/occupancy sensors to monitor the kitchen and eating areas and describe timing, frequency, and duration, including sensors for heat detection near a stove, on/off sensors for one or more kitchen appliances, light sensors to determine when lighting for the room or area is in use, sensors for drawer openings of a utensil drawer, door opening sensors for opening and closing of cabinets and pantries, etc. All of these sensors can be used to sense nominal levels to help the system establish a baseline of eating activities, so that when aberrant levels are sensed, or when otherwise normal sensed levels collectively exhibit aberrant patterns, anomalous activity may be discerned. It is appreciated that normal eating activities will likely differ based on time of day, day of week, existence of particular events (such as birthdays), etc., so any detection of anomalous activity will necessarily take such additional factors into consideration. The monitoring system 100 can also be configured such that indicators of other persons in the home are excluded from alerts based on the presence of an observer, such as one of the monitoring observers 106, who could determine the immediate needs of individual 102 without the assistance of monitoring system 100.

In some applications, the evaluation of eating activities can include the development of a “kitchen score.” As referred to herein, the “kitchen score” refers to a proprietary algorithm measuring the amount of effort spent preparing food in the kitchen with inputs from a plurality of different sensors and modalities, such as at least four different sensors and modalities in certain non-exclusive embodiments. The kitchen score can be used as a proxy for appetite, a component of assessment of the ability to self-care, and to detect food insecurity with a separate algorithm. Simple time measurement spent in the kitchen is not sufficient to understand eating patterns because some people do many things on kitchen working surfaces. Additionally, the term “meals” refers to an algorithm that learns the amount and timing of food-related activities for each home in order to show meals on the dashboard. While the system may contain multiple sensors in the kitchen to detect a diversity of data, it is important to separate activity related to food from other activities undertaken in the kitchen.

FIG. 2 is a representative example of a tabular illustration of sleeping activities, eating activities, movement activities, and medical device readings relevant to the individual 102 (illustrated in FIG. 1), and temperature readings for the setting 104 (illustrated in FIG. 1), which can be incorporated within functions of the monitoring system 100 (illustrated in FIG. 1). More specifically, FIG. 2 is a representative example of a Home Life Record® 220 that can represent a collection and summary of various data 116 (illustrated in FIG. 1) that has been received and/or derived from the plurality of data sources 108 (illustrated in FIG. 1) as disparately positioned throughout and outside the setting 104 during use of the monitoring system 100. It is the home equivalent of an electronic medical record for daily life events that are relevant to the health status of the whole individual 102, such as sleeping, eating, and activity details, as well as medical device and environmental status readings. It is appreciated that some of the data 116, such as medical device readings, can be transmitted to and/or incorporated within the Home Life Record® 220 through use of an application programming interface. As referred to herein, an application programming interface (API) consists of a software interface that allows computers to talk to each other, and in this case, to transfer data in an organized way. It is appreciated that the automated data collection made possible through generation and use of the Home Life Record® 220 relieves the individual 102 and the monitoring users 106 (illustrated in FIG. 1) from tedious documentation, while ensuring accuracy.

By way of example, regarding sleeping activities of the individual 104, the Home Life Record® 220 shows the number of hours of sleeping, as well as gaps in the sleeping (of differing lengths, with those greater than three hours being considered as wake periods) for eight particular days, and averages for such numbers over the previous week and month. With regard to eating activities of the individual 104, the Home Life Record® 220 shows the number of meals and the kitchen score for those eight particular days, with averages for such numbers over the previous week and month. With regard to movement activities of the individual 104, the Home Life Record® 220 shows an overall score, as well as numbers during sleep, and average in-home activity and day versus night activity ratio for those eight particular days, with averages for such numbers over the previous week and month. The Home Life Record® 220 also shows hours away from home for those eight particular days, with averages for such numbers over the previous week and month. With regard to medical device readings for the individual 104, the Home Life Record® 220 shows values from a blood pressure monitor (including blood pressure and pulse readings), an oximeter (including SpO2 and pulse readings), and weight scale measurements for those particular eight days, with averages for such numbers over the previous week and month. High and low temperatures are also shown for the same days, with averages over the previous week and month.

It is appreciated, however, that the Home Life Record® 220 can be tailored in any suitable manner, and can incorporate any data deemed appropriate according to individual needs. For example, the monitoring system 100 can generate data related to dementia, including hours away from home as a proxy for socialization, and day versus night activity ratio to quantify sundowning. The Home Life Record® 220 can further list counts of specific repetitive activities such as a number of times the refrigerator opens and closes, lights are turned on and off, doors are opened and closed, and/or the number and amount of time for bathroom visits during the day and during the sleep period. Other environmental sensor data would be reported similarly to the temperature reading, such as the high and low numbers of each day. Additionally, a behavioral matrix factor analysis can be used that would then test, for example, whether periods of high repetitive activity measurements or sundowning correlate with environmental factors like temperature and air quality being out of the normal or optimal range.

Based on the Home Life Record® 220, as used within the monitoring system 100, the monitoring system 100 can include any suitable number of types of alerts that can be sent out to the monitoring users 106 when anomalous activities or conduct are detected. In some embodiments, the monitoring system 100 can include up to 22 types of alerts, although the number can vary. Some types of alerts are based directly on sensor data, such as whether the refrigerator door or home exit door have been opened, or the home temperature. The data is used in a software program, or a “state machine” that places the thing to be measured in one of the defined conditions, in other words its state of being, e.g., active in home, inactive in home, sleeping, or away from home. Other alerts are based on a machine learning algorithm, such as the normal time the person gets out of bed for each day of the week (recognizing that some days, such as Sundays and Mondays in some instances, can be different), the kitchen score of effort spent on preparing a meal, or an inactivity alert that uses a state machine to understand whether the person is at home, and then scan for activity within a given time frame. This inhibits false alarms from lack of activity due to sleeping, or when the monitored individual 102 has left home. Each of these alerts can be passed on to the monitoring users 106 in any suitable manner, with reliability checks built in so that the monitoring users 106 are not alerted unnecessarily. Additionally, in certain embodiments, the monitoring system 100 can provide cellularly enabled medical devices and report when readings are out of range. Other alerts are possible based on other devices.

The monitoring system 100 can further incorporate other types of alerts and/or notifications. For example, a daily sleep notification can be sent out to the monitoring users 106 stating the time the individual 102 started sleep, the time sleep ended, and the total sleep hours. This notification would typically be sent out 30 to 60 minutes after rise time is confirmed. Such a notification of normalcy is usually desired by family care givers but not by clinical care givers who only want to be given information when anomalies occur. Therefore, the monitoring observer 106 should be able to select both the thresholds and the alerts/notifications that specific observer prefers. Additionally, environmental notifications can be reported in a timely manner if an environmental reading is out of range. For example, an out of range house temperature, air quality reading, noise decibel level, or other reading from the individual's personal environment can be included.

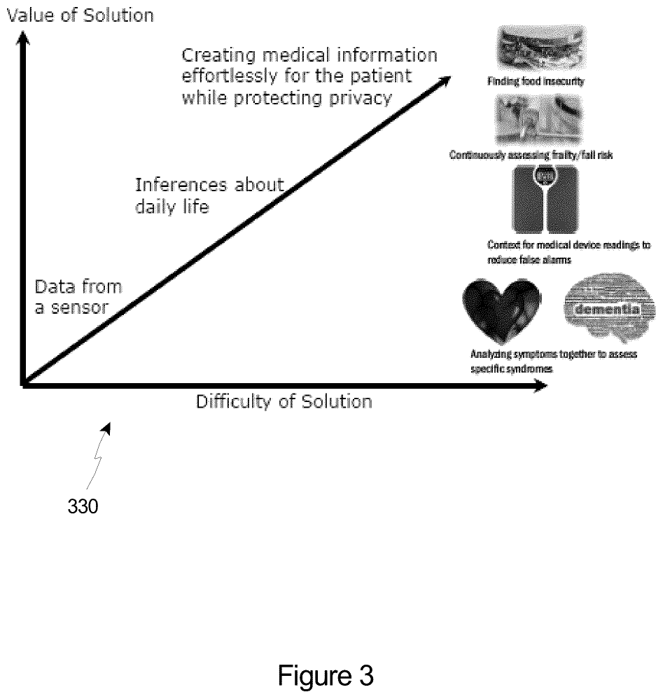

FIG. 3 is a simplified graphical illustration showing the general process for use of the monitoring system 100 (illustrated in FIG. 1), including analyzing symptoms clustered together to assess specific syndromes. More particularly, FIG. 3 is a simplified graphical illustration 330 with a value of solution shown along the Y-axis, and a difficulty of solution shown along the X-axis.

As illustrated in FIG. 3, the monitoring system 100 goes beyond reporting sensor data and basic inferences about daily life. Algorithms and behavioral analyses can create medical information effortlessly for the home resident by using passive sensors to collect strategic data, then applying that information to address specific problems. For example, food insecurity can be measured with the kitchen score, and frailty and fall risk can be continuously reviewed with movement assessment when the individual 102 (illustrated in FIG. 1) is alone versus when a helper is present. Additionally, algorithms and behavioral analyses that create context for medical device readings can interpret those physiological readings with environmental cues (e.g., weight gain from water retention vs. weight gain from having recently eaten a meal) thereby reducing false alarms. Further, algorithms and behavioral analyses can analyze symptoms together to assess when indicators of a disease episode are moving in the same direction. As illustrated and described herein below, this assessment of concurrent symptoms (also sometimes referred to as condition parameters or variables) can be expressed graphically as a visualized compilation data set such as a symptom cluster chart.

FIG. 4 is a simplified graphical illustration showing a visualized compilation data set 440, in the form of a symptom cluster chart, which can be used within the monitoring system 100 (illustrated in FIG. 1) to provide information about the individual 102 (illustrated in FIG. 1) to the monitoring users 106 (illustrated in FIG. 1). In particular, in various embodiments, the visualized compilation data set 440, or symptom cluster chart, can be displayed to the monitoring users 106 through use of the GUI 114 (illustrated in FIG. 1), which forms a part of the overall monitoring system 100. The visualized compilation data set 440, or symptom cluster chart, can also sometimes be referred to as a radar chart, a polar chart, or a star chart.

As shown, in many embodiments, a default setting for the visualized compilation data set 440, or symptom cluster chart, can include a single input axis being used for each element, variable, or condition parameter of the cluster. This provides a natural transition (as a condition parameter is added or removed) of the density and complexity of the graphical display, according to the number and possibly combinatorial isolation or overlap, of the selected element(s), variable(s), and condition parameter(s). With each specific symptom forming one ‘dimension’ (or input axis) of the display, the selection of symptoms to be co-displayed (and reviewed by the monitoring users 106) can be selected by the monitoring users 106 within the GUI 114 according to their choice of combinations, which may be specific to a described, or suspected, syndrome, and/or which can be a combination of associated symptoms, a characteristic combination of physiological readings, emotions and behaviors, or a joining of any set (or subset) of inputs integrated by the monitoring system 100, expressed by the individual 102, and observed by the monitoring user 106.

As illustrated in FIG. 4, the visualized compilation data set 440 can be utilized within the monitoring system 100 to explain and condense multiple variables (seven separate variables are shown in FIG. 4, labeled as “Variable A,” “Variable B,” “Variable C,” “Variable D,” “Variable E,” “Variable F,” and “Variable G”) into one graphic. The key features are that the visualized compilation data set 440 can include as many input variables (or condition parameters) as desired depending on the particular condition being reviewed, with each variable having its own input axis and scale and supporting data, and the placement of data on each input axis can be connected into a geometric shape (polygon). In many embodiments, the visualized compilation data set 440 will include at least three variables. For example, in certain non-exclusive implementations, the visualized compilation data set 440 can include three, four, five, six, seven, eight, nine, or ten variables. As described herein, color (including hue, brightness, intensity, shading, etc.), pattern, regularity/shape, and area can also be used to provide information about the polygon.

The goal for use of the visualized compilation data set 440 is to make oversight easier for the monitoring users 106. When the monitored individual 102 has stable daily data, the monitoring users 106 can see a small green polygon to indicate normalcy. When changes occur, the data building the polygon reflects those changes in the polygon size, shape, color (including hue, brightness, intensity, shading, etc.), and labels. However, it should not be necessary for the monitoring users 106 to have to view the polygon for the information to be meaningful and get the attention of the monitoring users 106.

It is appreciated that building the variable axes requires an understanding of thresholds for each variable, as described above, at which change (or deviation outside normal ranges) becomes relevant. When data is detected that will be placed outside the threshold of normal, an alert and/or notification can be automatically sent to the monitoring users 106. Examples include behavioral data, such as a delayed rise time, or an environmental measurement such as an inside temperature outside of normal, or a system problem like a low battery, or a medical device reading out of range. These thresholds could be raw data, such as the temperature reading, or derived data, such as recognition of the normal time range a person gets out of bed in the morning. The alert and/or notification could have a 2-way communication link that tracks whether and when the alert is communicated to the monitoring users 106, and whether, when and how the monitoring users 106 respond.

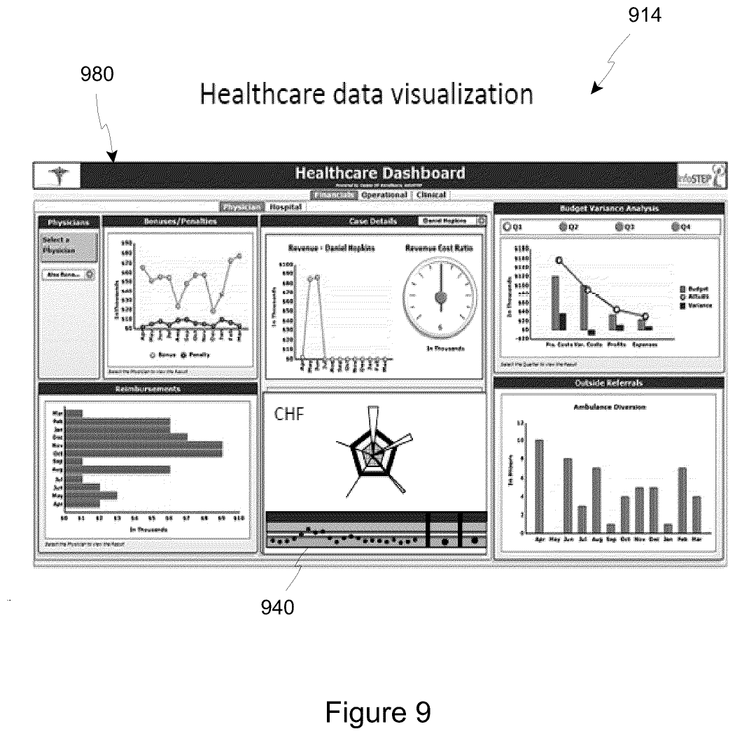

The visualized compilation data set 440 itself can also be shown in a report (such as the healthcare data visualization dashboard 980 shown in FIG. 9) and have automated text associated with current or trending variable data. Data and information could be visible, available in a popup, or dynamically controlled by algorithms. For example, if the visualized compilation data set 440 contains a variable whose input axis shows data outside the threshold for normal, the visualized compilation data set 440 could (1) state the current data value and the normal value, (2) compare to previous time periods like a prior week or month, or show trending data, (3) state comparison to a disease or wellness condition, (4) comment whether the change is trending toward or away from normal, (5) state whether and/or when the monitored individual 102 has generated this data value previously, (6) recognize potential for moving the data toward normal values, etc.

It is further appreciated that many systems automatically track the time an observer, such as a monitoring user 106, spends looking at a page, and where the observer goes next. Notes recorded at the time the visualized compilation data set 440 is observed, with action taken and results, would show people concerned with health and safety of the monitored individual 102 how much time was spent on each person.

For an open architecture system such as is incorporated within the present invention, there are optional sensors available as needed for a monitored individual 102. The visualized compilation data set 440 could therefore also be assembled from a selection of variables related to the sensors used by each monitored individual 102. The visualized compilation data set 440 can be focused to reflect symptom clusters specific to disease conditions, preferences of the monitored individual 102 or the monitoring users 106, and specialized needs. Each variable can therefore be added or subtracted from the visualized compilation data set 440 for personalization, clarity, and to drill down on which variables form an interactive cluster and which are independent based on parameters such as change of the polygon shape when a variable is added or subtracted.

Moreover, similar to home automation in which sensor readings will initiate an action (e.g., detection of room motion turns on a light), such interventions can be automated for data within the visualized compilation data set 440. For example, if a monitoring user 106 constructs the visualized compilation data set 440 for a specific individual 102 in which home temperature and toileting are both variables, if the visualized compilation data set 440 shows high temperature and reduced toileting, it could be associated with dehydration of the monitored individual 102. An automated intervention in this example could include a text or call to the monitored individual 102 with a reminder to drink a glass of water. A monitoring user 106 could therefore build a personalized visualized compilation data set 440 (or symptom cluster chart) with indwelling automated feedback based on the contained variables and the known or expected needs of the monitored individual 102.

It is also appreciated that for some variables in which a normal value lies in a range within a continuum, abnormal values are possible that are both less than and greater than normal. An example is weight, in which loss and gain can each be problematic. Another example is sleep duration, in which short sleep time and excessively long sleep times can both be symptoms of problems. Thus, strategies to visualize two-sided variables can include (i) for a static visualized compilation data set 440 (for example, printed on paper), the left and right side of the axis could specify gain or loss, and (ii) for a dynamic visualized compilation data set 440 (for example, on a screen), the trend could cause text to appear that specified directionality. For example, the variable axis could be labeled “weight” and the modifiers “gain” or “loss” could appear as specified by an algorithm based on data inputs, or be independent variable axes. Alternative solutions may also be employed when dealing with two-sided variables.

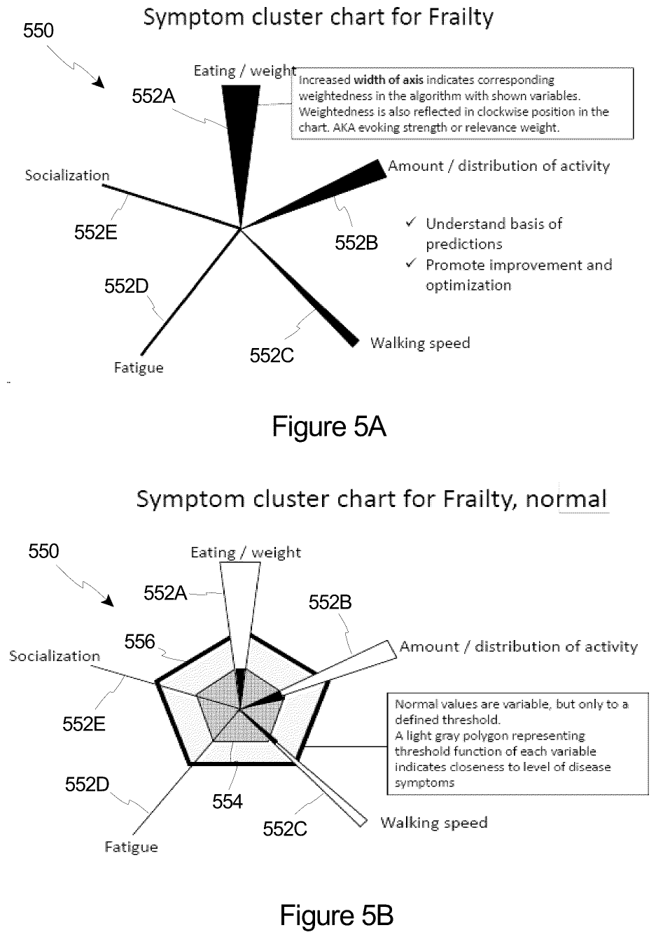

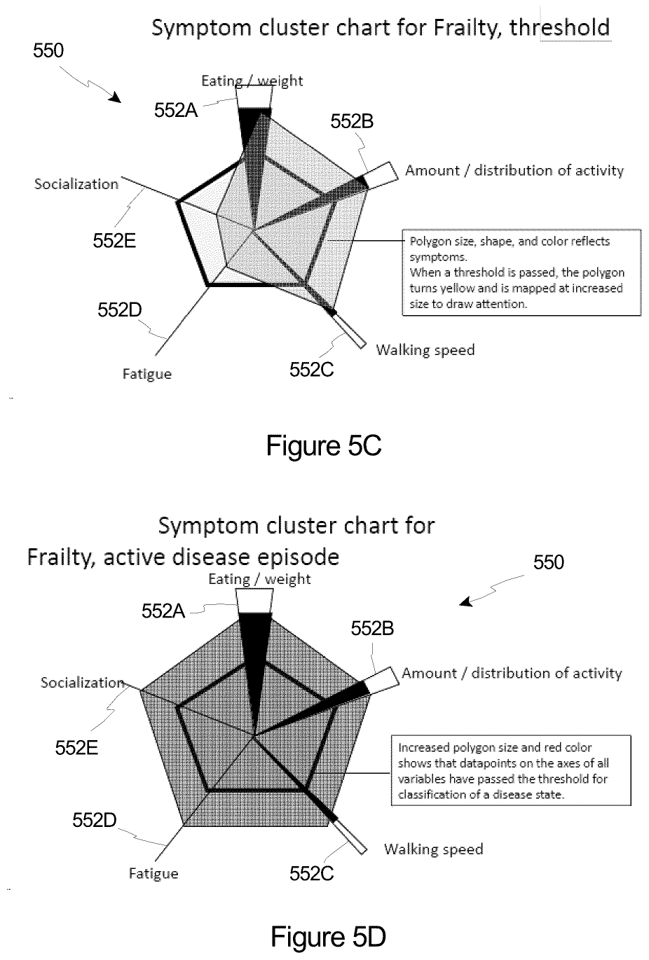

FIG. 5A is a simplified graphical illustration of factors usable within a symptom cluster chart 550 when reviewing potential frailty of the individual 102 (illustrated in FIG. 1) with the monitoring system 100 (illustrated in FIG. 1). As illustrated in FIG. 5A, the symptom cluster chart 550 includes five variables (or inputs) with potential relevance to frailty, namely, eating/weight, movement activity, walking speed, fatigue, and socialization. These named variables or inputs could also have their own indicators, such as activity described by time of day, amount, distribution, or other parameters. However, it is appreciated that the review of the individual 102 for potential frailty can include more variables or fewer variables than those specifically listed, and/or the specific variables can be modified in any suitable manner.

As shown, in this interpretation of the review of the individual 102 for potential frailty, each variable is labeled with a different level of importance within an appropriately designed algorithm, which is understood as being defined by a width of the input axis for the particular variable. In particular, a first variable 552A (for eating/weight) has the thickest input axis, a second variable 552B (for amount/distribution of activity) has the second thickest input axis, a third variable 552C (for walking speed) has the third thickest input axis, a fourth variable 552E (for fatigue) has the fourth thickest input axis, and a fifth variable 552E (for socialization) has the thinnest input axis. It is appreciated that the appropriate weight of the particular variable 552A-552E is also shown in FIG. 5A by its position on the star, with the highest weight variable being placed at 12:00 on an analog clock, and variables of decreasing weight being spaced apart from one another in a clockwise direction about the star. With such design, when shown to a monitoring user 106 (illustrated in FIG. 1), the listing of variables and their importance creates transparency for review based on the information and allows the monitoring user 106 to manipulate the variables to look for improvements or optimizations in how the individual's data fits the condition being reviewed by the monitoring user 106. For example, a potential modification to the algorithm can be addressed by determining if increasing and/or decreasing the weight assigned to any of the variables will help to define a better fit the known circumstance. Thus, it is appreciated that during actual use and implementation of the algorithm for the specifically identified condition, the weight assigned to each of the variables can be potentially modified to better understand the basis of review, as well as promoting improvement and optimization of the particular algorithm being used.

FIG. 5B is a simplified graphical illustration showing normal values and threshold values for each of the variables 552A-552E usable within the symptom cluster chart 550 when assessing conditions consistent with frailty of the individual 102 (illustrated in FIG. 1) with the monitoring system 100 (illustrated in FIG. 1).

In a first step, the data analytics resource 112 (illustrated in FIG. 1) can determine a normal range for each variable 552A-552E, which can each relate to an expected parameter state for a condition parameter based on data 116 (illustrated in FIG. 1) received from each of the plurality of data sources 108 (illustrated in FIG. 1) relative to the individual 102 within the setting 104 (illustrated in FIG. 1) over a predetermined period of time. To increase detail, the input axis of a variable can be open, and can be filled to the point on the axis representing the data point. The width of the input axis can also be marked to show relative weight, as noted above.

The normal range for each variable 552A-552E can then be plotted along the appropriate input axis, with each normal data point being a same distance from an origin of the star along a corresponding input axis, such as approximately one-third of the way along the input axis from the origin in one non-exclusive example. The data points of all input axes can then be connected to provide a regular polygon 554 shape. In this particular example, with five variables 552A-552E being used, the regular polygon 554 shape is a regular pentagon. During usage of the symptom cluster chart 550 for evaluating a given condition of the individual 102, a polygon connecting normal input values can be colored, such as green, in a manner to indicate the normalcy. The normal range for each variable 552A-552E can be determined in any suitable manner such as described herein above.

In addition to a given measurement, the monitoring users 106 (illustrated in FIG. 1) often want to know how that measurement compares to a threshold of concern. Thus, subsequently, the data analytics resource 112 can utilize an appropriate algorithm to determine a threshold for each variable 552A-552E specifying acceptable deviation from the normal range for the expected parameter state for each of the sensed condition parameters relative to the individual 102 within the setting 104. The input axes for each variable 552A-552E are therefore calibrated so that normal measurements are toward the center of the symptom cluster chart 550 (e.g., one-third of the way along the input axis from the origin), and the threshold of concern for each input axis is farther away from the center of the symptom cluster chart 550 (e.g., two-thirds of the way along the input axis from the origin). As shown, the threshold value for each of the variables 552A-552E will also typically provide a regular polygon 556 shape (a pentagon in this particular example as it includes five variables 552A-552E) by connecting the threshold values for each variable 552A-552E as plotted along the appropriate input axis at the same distance from the center point, or origin.

It is appreciated that since the threshold of concern for each input axis is farther away from the center, or origin, of the symptom cluster chart 550 than the normal range of measurements for each variable 552A-552E, the regular polygon 556 shape encompassing the threshold value for each of the variables 552A-552E will be somewhat larger than the regular polygon 554 shape encompassing the normal range of measurements for each of the variables 552A-552E.

FIG. 5C is a simplified graphical illustration showing actual values for each of the variables 552A-552E usable within the symptom cluster chart 550 when reviewing frailty of the individual 102 (illustrated in FIG. 1) with the monitoring system 100 (illustrated in FIG. 1). As noted above, after determination of the normal range and threshold acceptable deviation therefrom for each variable 552A-552E (for each expected parameter state for each condition parameter), the data analytics resource 112 (illustrated in FIG. 1) is configured to receive at least one additional data point from each of the plurality of data sources 108 (illustrated in FIG. 1). The at least one additional data point can then be plotted directly, or through appropriate derivation in instances in which multiple points of data are combined in a multifactorial fashion to infer status of the variable, or condition parameter, onto the symptom cluster chart 550 so that the monitoring users 106 (illustrated in FIG. 1) can see how far the measurement from the at least one additional data point is from the threshold of concern for each variable 552A-552E.

As shown in FIG. 5C, when one or more of the variables 552A-552E have data points that have passed the threshold for that input axis, the polygon can change in one or more of size, shape and color. For example, in such a situation, the polygon can change to a different color, such as yellow, and the size of the polygon can increase. As also shown in FIG. 5C, if the variables 552A-552E are not tracking together (i.e. they are not increasing with the same magnitude at the same time interval), the yellow polygon will become asymmetric or irregularly shaped. The increased size, and change in shape and color, is intended to attract the attention of the monitoring users 106. It is appreciated that these transitions can also be recorded as time series data to feed algorithms and analyses. The color can be transparent enough to show where the threshold is for each axis, and therefore how far the current data point is from the threshold of concern.

FIG. 5D is another simplified graphical illustration showing actual values for each of the variables 552A-552E usable within the symptom cluster chart 550 when reviewing frailty of the individual 102 (illustrated in FIG. 1) with the monitoring system 100 (illustrated in FIG. 1). In the example shown in FIG. 5D, the symptom cluster chart 550 shows that measurements from additional data points in all variables 552A-552E have passed the threshold of concern. More specifically, as shown, each of the variables 552A-552E have passed the threshold of concern and are tracking together (i.e. they are increasing with the same magnitude at the same time interval), so as to generate a third, even larger, regular polygon. In such situations, the appropriate algorithm can utilize a second threshold to determine when to transition the color of the polygon from yellow to a third color, such as red. In one particular example, such as for blood pressure values, numbers within 10 points of normal could be yellow, while numbers more than 10 points from the initial threshold could turn the polygon red.

As shown, in this example implementation, the polygon color is red, and the area is enlarged toward the top of each of the input axes. A red star will approach maximal size to attract attention for monitoring users 106 (illustrated in FIG. 1) using the visual impact of both area and color.

It is further appreciated that calculating the area of the star polygon is an easily trackable summary of the individual's condition. The smaller the area of the polygon, the more normal the symptoms are. An algorithm to create the reported value of the polygon area can be simple, such as just area covered, or based on an algorithm that takes into account the weight of each variable 552A-552E, proximity to threshold, recent changes, similarity of progression from the center of a number of variables, etc.

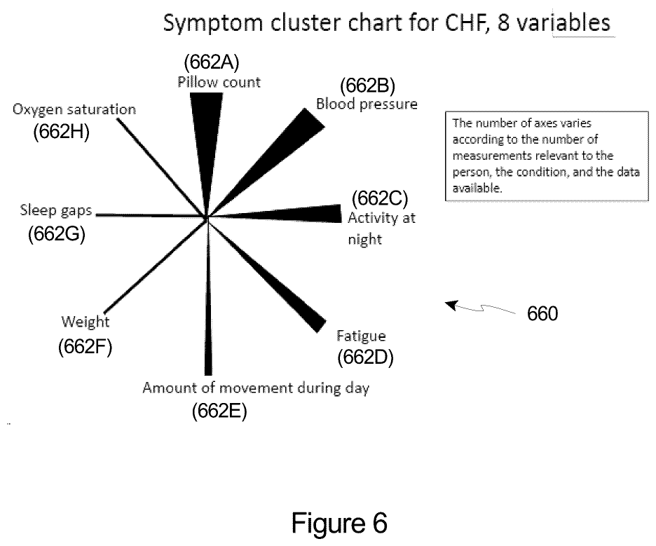

FIG. 6 is a simplified graphical illustration of factors usable within a symptom cluster chart 660 when reviewing consistency of symptoms with congestive heart failure of the individual 102 (illustrated in FIG. 1) with the monitoring system 100 (illustrated in FIG. 1). As shown, in this interpretation of the review of the individual 102 for potential congestive heart failure, the symptom cluster chart 660 includes eight variables (or inputs), with each variable again being labeled with a different level of importance within an appropriately designed algorithm, which is again understood as being defined by a width of the input axis and a clockwise positioning of the particular variable. However, it is appreciated that the review of the individual 102 for potential congestive heart failure can include more variables or fewer variables than those specifically listed, and/or the specific variables can be modified in any suitable manner.

In particular, listed from most weighted to least weighted, the symptom cluster chart 660 includes a first variable 662A (for pillow count, used on the bed to prop up the individual's torso to help breathe during sleep), a second variable 662B (for blood pressure readings), a third variable 662C (for activity at night), a fourth variable 662D (for fatigue), a fifth variable 662E (for amount of movement during the day), a sixth variable 662F (for weight), a seventh variable 662G (for sleep gaps), and an eighth variable 662H (for oxygen saturation). It is appreciated that a monitoring user 106 (illustrated in FIG. 1) can again add or subtract variables, and increase or decrease weight for each variable, to see what could optimize the fit of the symptom cluster chart 660 to the individual's circumstance, with the goal being to improve the comparative analytics and personalization of the symptom cluster chart 660.

Although not shown in FIG. 6, the symptom cluster chart 660 would also include regular polygons (i.e. octagons in this particular example that includes eight variables 662A-662H) relating to (i) a normal range for each of the variables 662A-662H as determined by the data analytics resource 112 (as illustrated in FIG. 1), upon receiving data 116 (illustrated in FIG. 1) relevant to the expected parameter state for the condition parameter (i.e. the variable 662A-662H) from each of the plurality of data sources 108 (illustrated in FIG. 1) relative to the individual 102 within the setting 104 (illustrated in FIG. 1) over a predetermined period of time; and (ii) a threshold of acceptable variation from the normal range for each of the variables 662A-662H as determined by the data analytics resource 112.

Also similar to as described above in relation to FIGS. 5A-5D above, the symptom cluster chart 660 would also include a polygon that is based on at least one additional data point that is received by data analytics resource 112 relevant to each of the variables 662A-662H from each of the plurality of data sources 108. This subsequently developed polygon could vary in terms of size, shape and color from the regular polygon (octagon) as the at least one additional data point for any of the variables 662A-662H extends outside the normal range and/or beyond the threshold. In one non-exclusive embodiment, as above, green can indicate normal conditions, yellow can indicate concern, and red can indicate a need for response. A further embodiment enables the user(s) to change the color display to account for potential user needs such as color blindness, social acceptability or disfavor for choices of particular color, or color patterns creating interpretive problems from display inefficiencies or unanticipated technical inaccuracies.

With the desired use of any appropriate symptom cluster chart 660, it is appreciated that when many symptoms (variables or condition parameters) are considered, just showing each symptom (variable or condition parameter) individually does not provide an easy understanding of the multitude of components of the individual's condition, progression, or needs. Additionally, as noted, some symptoms (variables or condition parameters) should be more strongly weighted than others, and the thresholds for concern should be determined, and whether the symptoms (variables or condition parameters) are moving together in one direction (better or worse) can be determined.

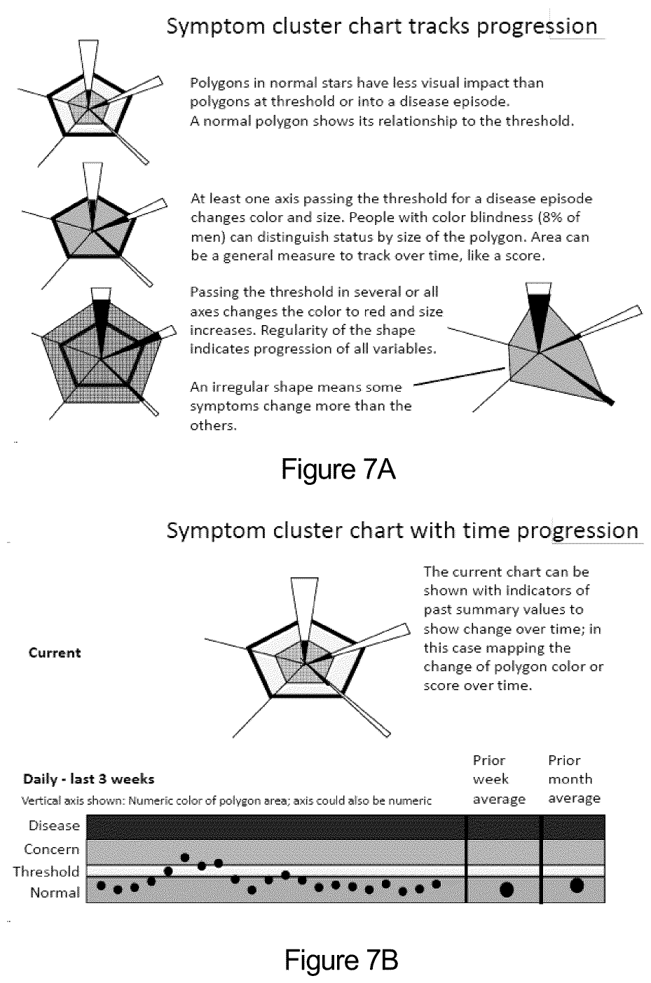

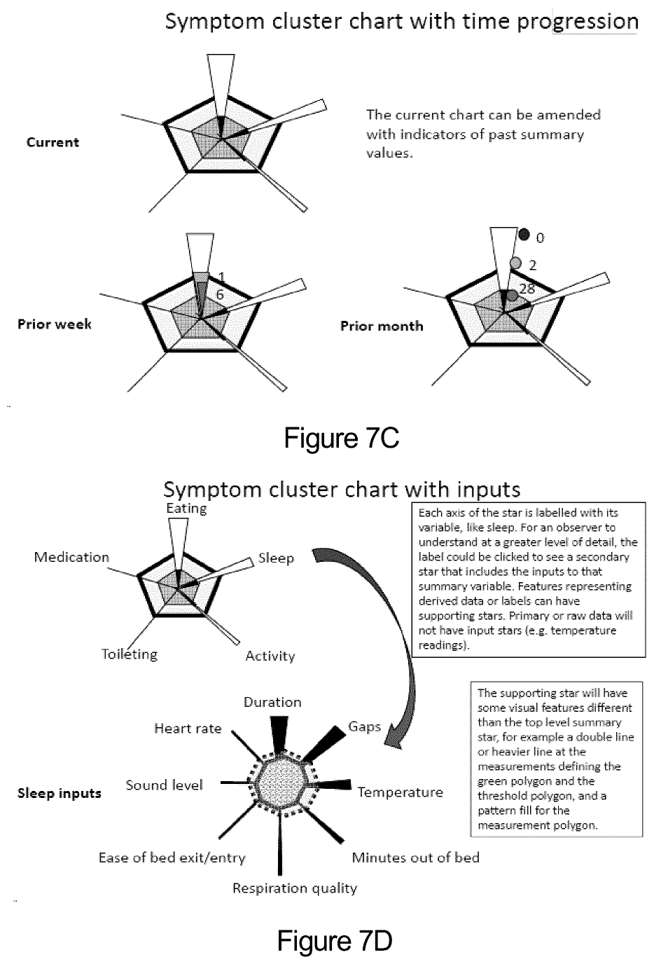

FIGS. 7A-7D are a series of simplified illustrations and descriptions regarding how the symptom cluster chart can incorporate and/or be updated to track trends and time progression for each of the factors (variables) included when determining consistency of any potential condition of the individual 102 with a given condition (illustrated in FIG. 1) with the monitoring system 100 (illustrated in FIG. 1).

For example, FIG. 7A is a simplified illustration and description of how the symptom cluster chart tracks progression of actual values for each of the factors included when determining consistency of any potential condition of the individual 102 (illustrated in FIG. 1) to a known condition with the monitoring system 100 (illustrated in FIG. 1).

As illustrated, FIG. 7A shows how the symptom cluster chart can be used to illustrate different characteristics relative to the similarity of the potential condition of the individual to a known condition. In particular, the symptom cluster chart can be shown with the polygon having a first color, such as green, to show normalcy, which would have less visual impact than other colors used if the factors (variables or condition parameters) are at threshold or into a disease episode. The regular (green) polygon clearly shows its relationship to the threshold.

Additionally, the symptom cluster chart can be shown with the polygon having a second color, such as yellow, when at least one factor (variable or condition parameter) extends beyond its threshold value. It is appreciated that as factor values approach and/or extend past the threshold value, the size of the polygon also changes, which can be another cue for the monitoring users 106 (illustrated in FIG. 1) in addition to the change in color. It is understood that the calculated area of the polygon can be reported and represents a summary measurement that can be tracked over time to monitor symptom progression or retreat.

Further, the symptom cluster chart can be shown with the polygon having a third color, such as red, when several or all of the factors (variables or condition parameters) have passed their threshold value. It is also noted that the size of the polygon increases correspondingly. Continued regularity of the shape of the polygon indicates that the factors are changing concurrently in similar magnitude.

Still further, in some instances, the symptom cluster chart will be illustrated as an irregular or asymmetrically-shaped polygon. It is appreciated that although a circumstance of concern can be characterized by specific variables, there are variables like fatigue or loss of appetite that are found in many different conditions. When the shape of a polygon whose inputs are consistent with a specific condition or disease is irregular, it shows that some variables are changing while others are not, or the changes in variables are not tracking with one another. This can provide the monitoring user 106 with clues as to whether the changes are related to the expected specific condition or whether a different set of symptoms could create a more regular polygon and therefore be consistent with another condition. A monitoring user 106 could potentially swap out variables to test whether a polygon can be created that shows change in a specific set of variables to recommend examining an alternative current condition. For example, both pneumonia and heart failure have loss of appetite, less activity, and disturbed sleep, but pneumonia could show weight loss and heart failure would show water weight gain.

FIG. 7B is another simplified illustration and description of how the symptom cluster chart tracks progression of actual values for each of the factors (variables or condition parameters) included when reviewing any potential condition of the individual 102 (illustrated in FIG. 1) with the monitoring system 100 (illustrated in FIG. 1). In the top portion of FIG. 7B, a “current” state version of the symptom cluster chart is shown. In the bottom portion of FIG. 7B, a chart is shown that includes indicators of past summary values to show change over time. In particular, the chart provides daily summary values for the last three weeks, as well as prior week and prior month averages. In certain implementations, the chart illustrating the previous values could track as shown for color, or could instead replace color with a numeric value of the polygon on the vertical axis (thus defining disease state, concern, threshold, and normal by polygon area). The defined time period of the prior week or month is also shown for longitudinal comparison. In the particular case illustrated in FIG. 7B, the area or color of the polygon is graphed left to right to illustrate polygon metrics with passage of time, or to depict an average value over a defined time.

FIG. 7C is still another simplified illustration and description of how the symptom cluster chart tracks progression of actual values for each of the factors (variables or condition parameters) included when reviewing any potential condition of the individual 102 (illustrated in FIG. 1) with the monitoring system 100 (illustrated in FIG. 1). As illustrated, there are alternative ways to describe time progression graphically. For example, when there is space constraint, numbers can be added to each input axis that indicate how many times in a defined period that factor (variable or condition parameter) showed a measurement within normal (green), concern (yellow), or disease (red) levels. As understood, each variable represents either direct sensor data or a value derived from its own inputs, and a symptom cluster chart can include both direct and derived variables at the same time. A monitoring user 106 (illustrated in FIG. 1) may want to understand a particular variable at a greater level of detail. The variable name could therefore link to a further symptom cluster chart (or star) that represents the specific inputs into that variable. An example of such greater level of detail is provided in FIG. 7D.

FIG. 7D is a simplified illustration and description of how one of the factors (variables or condition parameters) included within the symptom cluster chart can be further broken down into a plurality of factor inputs when reviewing a potential condition of the individual 102 (illustrated in FIG. 1) with the monitoring system 100 (illustrated in FIG. 1).

In the particular example shown in FIG. 7D, the primary symptom cluster chart is illustrated as including variables (in decreasing weight order) relating to eating, sleep, activity, toileting, and medication. To understand a greater level of detail relating to any of these factors, the monitoring user 106 (illustrated in FIG. 1) can simply click on the variable name to open up a further (or deeper) symptom cluster chart that illustrates the inputs for that particular variable. It is appreciated, however, the factors or variables on the primary symptom cluster chart that merely include primary or raw data cannot be opened up to a further (or deeper) symptom cluster chart.

As shown, by clicking on the “sleep” variable name, the further (or deeper) symptom cluster chart shows inputs into the sleep variable (in similar decreasing weight order) of duration, sleep gaps, room temperature, minutes out of bed through a sleep cycle, respiration quality, ease of bed exit/entry, room sound level, and heart rate. The further (or deeper) symptom cluster chart can also be used to develop additional factors such as a restlessness score. As with the primary symptom cluster chart, each variable in the further (or deeper) symptom cluster chart would have its own axis, its own threshold of concern, and its own polygon drawn from the represented axis data points. There may be a visual indicator that this is a supporting symptom cluster chart, so the polygon could have patterned fill or another visual indicator that it feeds a higher level symptom cluster chart. Additionally, the threshold could have a double line, a dashed line, or other different visual indicators. In some embodiments, it is generally preferred that the highest level symptom cluster chart have the simplest presentation. A series of supporting symptom cluster charts could then be implemented to drill down to primary sensor data from any variable that represents derived data.

FIG. 8 is a simplified flowchart demonstrating a system and method for generating a symptom cluster chart having features of the present invention. As described, the system and method can be used for monitoring at least one condition of an individual. As described herein, in various embodiments, the system and method for generating the symptom cluster chart is accomplished through use of the data analytics resource 112 (illustrated in FIG. 1).

It is recognized that in nonexclusive alternative embodiments, the system and method of FIG. 8 can include additional steps other than those specifically delineated herein or can omit certain of the steps that are specifically delineated herein. Moreover, in some embodiments, the order of the steps described below can be modified and/or certain steps can be combined without deviating from the spirit of the present invention.

At step 870, the system and method includes generating three or more input axes for the symptom cluster chart that extend outwardly from a common origin with the data analytics resource, each of the three or more input axes relating to a condition parameter of the at least one condition relevant to the individual.

At step 871, the system and method includes receiving data for each condition parameter relative to a predetermined period of time with the data analytics resource. In many embodiments, the data is received by the data analytics resource from a plurality of data sources, which are positioned disparately within or outside a setting in which the individual is located. As noted above, the plurality of data sources can include sensors, monitors, medical devices, measurement devices, medical records, or any other suitable data sources. Additionally, as further noted above, the condition parameters relevant to the at least one condition can be generated from discrete data from individual data sources, or can be derived from data received from any suitable combination of data sources.

At step 872, the system and method includes determining a normal range for each condition parameter based on the received data with the data analytics resource.

At step 873, the system and method includes determining a proportion of each input axis that can correspond to normal, threshold of concern, and warrants follow-up, relative to the at least one condition of the individual. Typically, the proportion used for each level along the input axes will be the same for each input axis, i.e. for each condition parameter. In one non-exclusive embodiment, a normal range can be plotted approximately one-third of the way along each input axis from the common origin, a threshold of concern can be plotted approximately two-thirds of the way along each input axis from the common origin, and a warranting follow-up level can be plotted at or near the outer end of each input axis (or anywhere outside the noted threshold of concern).

At step 874, the system and method includes assigning numeric value along the three or more input axes for each of the condition parameters of the at least one condition with the data analytics resource to reflect the relevant data so that the normal range for each condition parameter forms a first regular polygon shape when plotted and connected along each of the three or more input axes on the symptom cluster chart. So, for example, (1) if three input axes are used, the normal range for the condition parameters, when plotted and connected along each of the input axes would form a regular triangle; (2) if four input axes are used, the normal range for the condition parameters, when plotted and connected along each of the input axes would form a regular quadrilateral; (3) if five input axes are used, the normal range for the condition parameters, when plotted and connected along each of the input axes would form a regular pentagon; (4) if six input axes are used, the normal range for the condition parameters, when plotted and connected along each of the input axes would form a regular hexagon; (5) if seven input axes are used, the normal range for the condition parameters, when plotted and connected along each of the input axes would form a regular heptagon; and (6) if eight input axes are used, the normal range for the condition parameters, when plotted and connected along each of the input axes would form a regular octagon.

At step 875, the system and method includes establishing a threshold of acceptable deviation from the normal range for each condition parameter with the data analytics resource so that the threshold of acceptable deviation for each condition parameter forms a second regular polygon shape when plotted and connected along each of the three or more input axes on the symptom cluster chart, the second normal polygon shape being larger than the first normal polygon shape. In some embodiments, additional thresholds of acceptable deviation for the condition parameter could be graphically illustrated and an additional data point changes the symptom cluster chart in one or more of shape, size, fill pattern, and color. It is appreciated that the intent is to provide variations along each axis, such as a green, yellow, and red zone for an axis. For example, normal sleep, low sleep, no sleep. However, other possibilities can include more than three levels of variation, such as a five stage axis for blood pressure including normal, elevated, and hypertension stages 1, 2 or 3. In certain embodiments, the threshold of acceptable deviation can be associated with change in expected response of the monitoring users, such as a caregiver.

At step 876, the system and method includes receiving at least one additional data point for each condition parameter with the data analytics resource.

1 At step 877, the system and method includes plotting the at least one additional data point for each condition parameter along the three or more input axes.

At step 878, the system and method includes connecting the at least one additional data point for each condition parameter to form an updated polygon.

At step 879, the system and method includes graphically illustrating the symptom cluster chart on a graphical user interface. As noted above, when plotting and connecting the additional data points for each condition parameter along a corresponding input axis, the resulting figure can differ from the regular polygons representing the normal range and the threshold of concern in one or more of size, shape, color (including hue, brightness, intensity, shading, etc.). With the noted potential variations in size, shape and color, the monitoring users 106 (illustrated in FIG. 1) are quickly, clearly, and unambiguously notified or alerted when a level of concern is reached regarding the at least one condition of the individual to warrant follow-up.

As so described, the present invention enables a matching between not just a linear, or planar, but also a spectral display, wherein the transition from each ‘clearly dominant’ color through intermediate transitional shadings to the next dominant color, convey better a positional matching of the selected data reading(s) at each interval of observation and allow also a progression (or regression, retrogression, or devolution), according to the selections of intervals.

FIG. 9 is a representative example of a healthcare data visualization dashboard 980 (also referred to herein as a “healthcare dashboard) that can be illustrated within a graphical user interface 914, including an example of the symptom cluster chart 940 with time progression as shown in FIG. 7B. As illustrated, most of the graphics shown in the healthcare dashboard 980 are informative, but are not necessarily simple to understand for the monitoring users 106 (illustrated in FIG. 1). However, the visualized compilation data set in the form of the symptom cluster chart 940 provides graphics incorporating size, shape and colors that are clear and unambiguous, and can thus be interpreted quickly and easily by the monitoring users 106 in the manner as described in detail herein above.

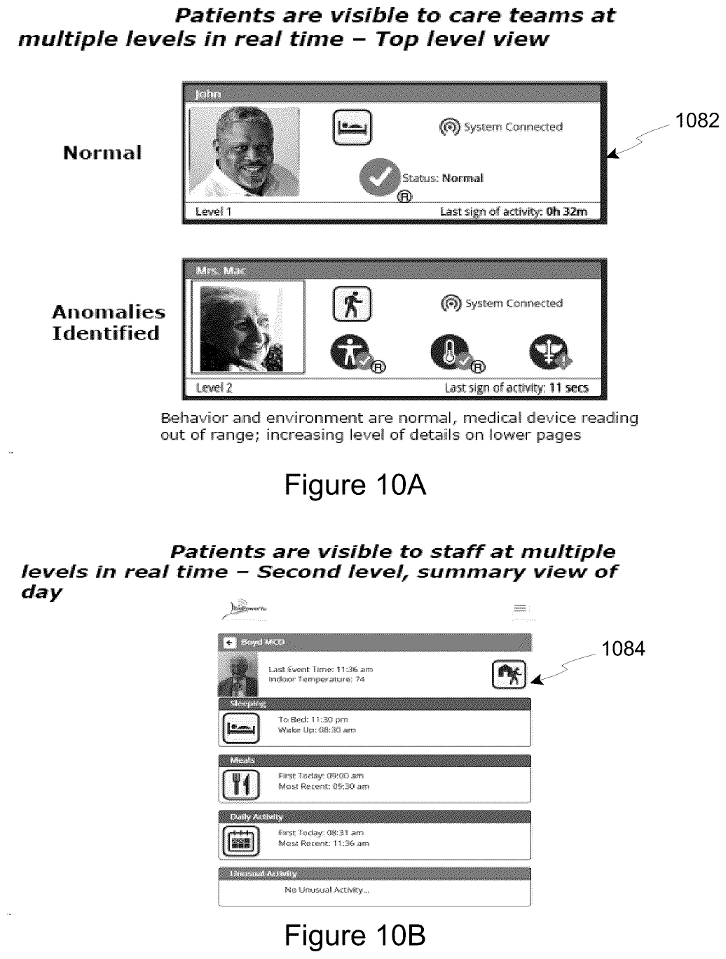

FIG. 10A is a simplified illustration of an example top level view of a general review chart 1082 of at least one individual (top level general evaluations are shown for two individuals in FIG. 10A) regarding one or more activities and environmental readings relevant to the individual that can be provided through use of the monitoring system 100 (illustrated in FIG. 1). More particularly, FIG. 10A shows a representative embodiment of a first graphic that can be seen by a monitoring user 106 (illustrated in FIG. 1) of a monitored individual 102 (illustrated in FIG. 1) when the monitoring user 106 signs in to the web-based dashboard of the present invention.

As shown, each monitored individual 102 is represented by a rectangular card with a banner showing current condition (such green for normal, orange/yellow for anomaly detected, and red for concern). The individual 102 can be identified by a short name, such as John or Mrs. Mac in this example, and optionally by an image such as a photo or drawn avatar. To the right of the visual image, directly below the banner, each card identifies the current home state with a square icon from four options: sleep (like John), active (like Mrs. Mac), inactive at home, or away from home. An icon and text can further be included that identifies that the monitoring system is connected and therefore that the data is current. Below the state and connectivity icons are icons comparing the monitored person's data to their personal baseline at a high level. As noted, all of John's data is normal, so he can have a relatively large checkmark within a green circle, and his banner can be green. However, the monitoring system detected an anomaly or out-of-range data for Mrs. Mac, so her banner can be orange/yellow and instead of one consolidated checkmark she can have three icons. The left icon represents behavioral data, which is normal and can be shown to be normal with a checkmark in a green circle. The middle icon represents environmental data, like temperature, and can be shown to be normal with a checkmark in a green circle. The right icon represents medical device readings, and an orange/yellow exclamation point can indicate that the most recent reading is out of the defined normal range. Therefore, Mrs. Mac's banner can be orange/yellow. Both cards also state the time of the last detected sensor event for sensors triggered by the monitored individual 102 (as opposed to sensor readings like temperature whose changes are unrelated to home activity).

FIG. 10B is a simplified illustration of an example second level, summary view of a general review chart 1084 of the individual regarding one or more activities and environmental readings relevant to the individual that can be provided through use of the monitoring system 100 (illustrated in FIG. 1). In this example, it is appreciated that information from the top-level view is carried down to the second level general review chart 1084, with additional details. The banner, monitored person identification (“Boyd MCD” in this particular example), and home status (in this case showing away from home), are repeated. Additional information shown in this second level general review chart 1084 includes current home temperature, and details related to sleeping, meals, daily activity, and anomalies (unusual activity). Buttons to access other pages related to Settings, the Home Life Record®, External Connections, and Battery Levels can also be provided. Additionally, in many embodiments, the rectangles containing information on sleeping, meals, daily activity, and unusual activity can be clicked on to see additional information at a lower (third) level.

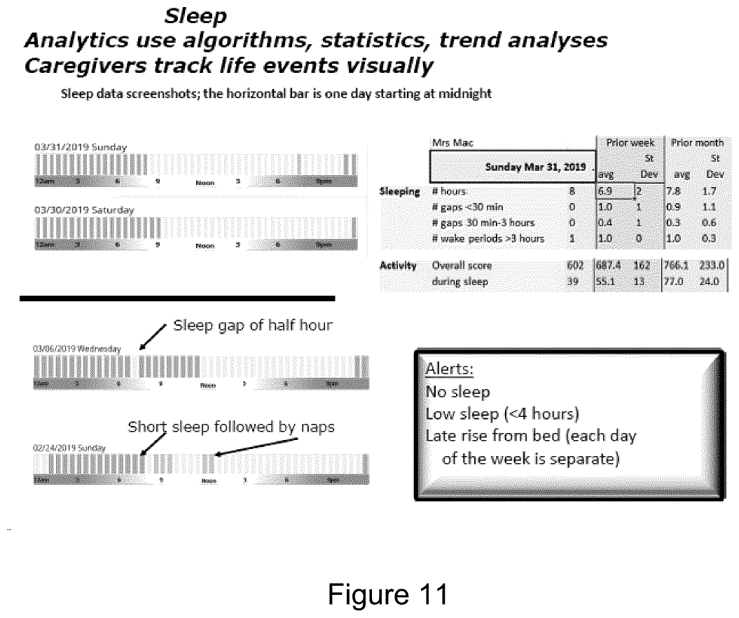

FIG. 11 is a series of related graphical illustrations usable to review sleep activities of the individual 102 (illustrated in FIG. 1) that can be provided through use of the monitoring system 100 (illustrated in FIG. 1). As described herein, these measurements can include discrete data or derived information that is presented in the symptom cluster chart related to sleep. sleep time, wake time, sleep duration, and sleep gaps as sensed or inferred by the plurality of data sources 108 (illustrated in FIG. 1)

In particular, the graphical illustrations incorporate sleep analytics that use algorithms, statistics and trend analyses that, when viewed by the monitoring users 106 (illustrated in FIG. 1), enable the monitoring users 106 to track life events, such as sleep episodes, of the individual 102 visually. It is appreciated that such graphical illustrations can encompass a lower (third) level detailed view of sleeping patterns, in this particular example. Such details can also be provided in any suitable manner within the Home Life Record® 220 (illustrated in FIG. 2) and/or the healthcare dashboard 980 (illustrated in FIG. 9).

As shown in FIG. 11, each day is a horizontal bar divided into half hour columns. For example, the monitoring users 106 can see when the monitored individual 102 entered the bedroom and began a period of quiet (the inference of sleep). The time mapping of the sleeping patterns can show a colored (such as blue) column in each half hour in which activity level meets the inference engine criteria for “sleep.” Examples of uninterrupted nights of sleep are shown on the top left. Sequential periods of sleep, and short periods of inactivity in the bedroom (naps) are shown on the bottom left. An example of how that data could appear on the Home Life Record® 220 is shown on the top right. Potential alert responses by the monitoring system 100 are shown on the bottom right. The inference engine and response system can therefore be programmed to detect and alert the monitoring users 106 to multiple types of activities or events related to inferred sleep.

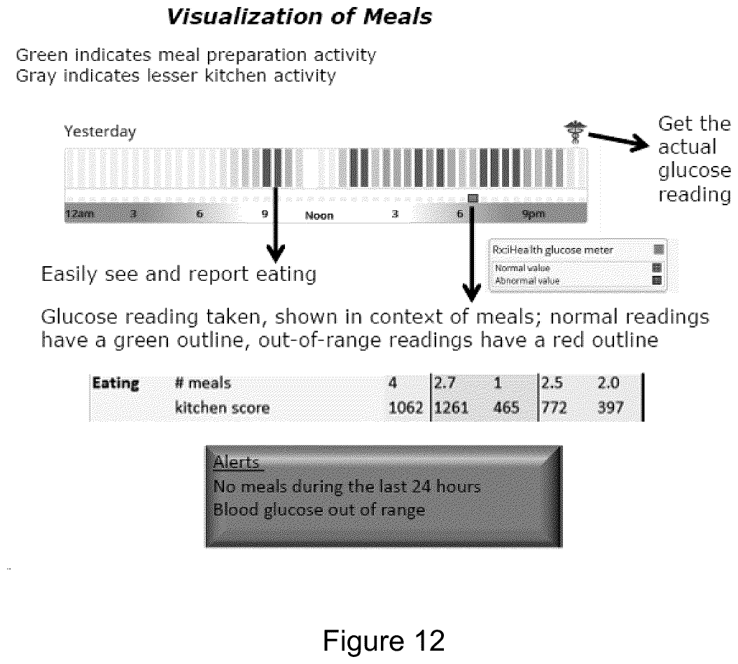

FIG. 12 is a series of related graphical illustrations usable to review eating activities of the individual 102 (illustrated in FIG. 1) that can be provided through use of the monitoring system 100 (illustrated in FIG. 1). As described herein, these measurements can include discrete data or derived information that is presented in the symptom cluster chart related to eating.

In particular, the graphical illustrations incorporate eating analytics that use algorithms, statistics and trend analyses that, when viewed by the monitoring users 106 (illustrated in FIG. 1), enable the monitoring users 106 to track life events, such as meals, of the individual 102 visually. It is appreciated that such graphical illustrations can encompass a lower (third) level detailed view of eating activities or meals, in this particular example. Such details can also be provided in any suitable manner within the Home Life Record® 220 (illustrated in FIG. 2) and/or the healthcare dashboard 980 (illustrated in FIG. 9).

In several embodiments, the inference engine can use data sources 108 (illustrated in FIG. 1) placed in the kitchen to infer meals and can further show the timing of those meals on the dashboard page labeled “Meals.” Activity at the level defined as “meals” can be shown in a particular color (such as green) during the half hour in which it was produced. In this particular example, meals have been detected at times that would coincide with typical breakfast, lunch, afternoon snack, and dinner activities. Activity that does not meet the criteria of “meals” can be shown in a different, more muted color, such as gray, with light gray potentially indicating a lower level of non-meal activity than darker gray.

Additionally, there are common medical devices associated with eating, such as a blood glucose meter for individuals with diabetes. Because of the value of understanding the timing and results of blood glucose readings in relation to meals, the visualized compilation data set, such as the symptom cluster chart, as incorporated within the invention, can be configured to show when a reading from a linked blood glucose device is taken, the data accessed by API and transferred to the data collection resource 110 (illustrated in FIG. 1) and/or the data analytics resource 112 (illustrated in FIG. 1) for presentation on the GUI 114, and gives it a positive appearance, such as a green outline, when the reading is normal. Conversely, if the reading is outside of the normal range defined within the settings of the symptom cluster chart, the reading can get a more negative appearance, such as a red outline. The abnormal reading can then show up on the top-level healthcare dashboard 980 as an orange or red banner, depending on how far from the normal range the reading value is.

In some embodiments, the monitoring users 106 can see a medical icon on the top right corner of any day in which a medical device reading has been taken. In certain embodiments, if the monitoring user 106 clicks on the medical icon, a page with all medical data from the current day will appear.

Similar to the sleep page, the data collected regarding these eating activities can be shown on the Home Life Record® 220, in this case the number of meals and the kitchen score. Possible alerts from the monitoring system 100 related to meals, as shown, can include whether any meals were detected in the last 24 hours and whether an associated medical device reading is out of range. However, it is appreciated that other suitable inferences and alerts are also possible.

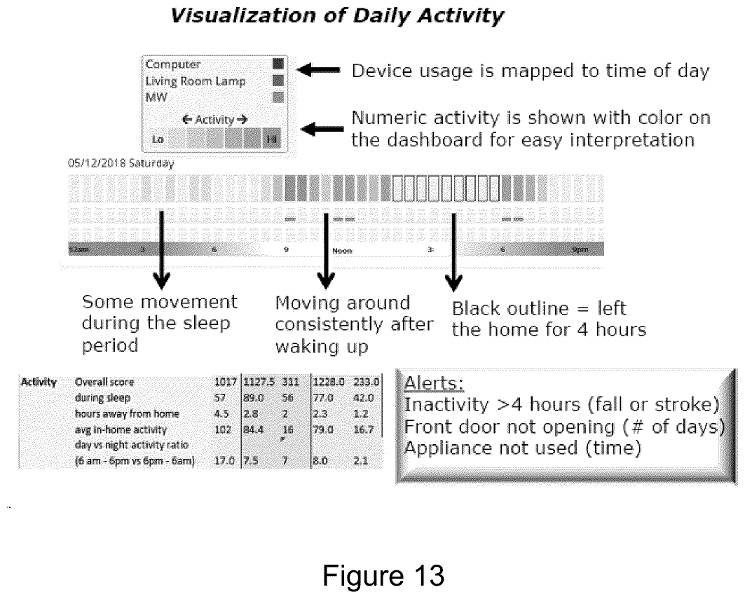

FIG. 13 is a series of related graphical illustrations usable to review movement activities of the individual 102 (illustrated in FIG. 1) that can be provided through use of the monitoring system 100 (illustrated in FIG. 1). As described herein, these measurements can include discrete data or derived information that is presented in the symptom cluster chart related to movement related to the home.

In particular, the graphical illustrations incorporate movement analytics that use algorithms, statistics and trend analyses that, when viewed by the monitoring users 106 (illustrated in FIG. 1), enable the monitoring users 106 to track life events of the individual 102 visually. It is appreciated that such graphical illustrations can encompass a lower (third) level detailed view of movement activities, in this particular example. Such details can also be provided in any suitable manner within the Home Life Record® 220 (illustrated in FIG. 2) and/or the healthcare dashboard 980 (illustrated in FIG. 9).

As shown in FIG. 13, a time map is included illustrating distribution and amount of movement activity by the individual 102 in the setting 104 (illustrated in FIG. 1) each day. It is appreciated that specific sensors, including motion sensors, are linked to this analysis, and when activity is detected, the half hour interval in which the activity occurs becomes colored. In one non-exclusive embodiment, low levels of activity can be shown on the map as light yellow, and as the amount of activity within the half hour increases, the color can transition in steps to dark orange representing activity throughout the half hour.

Devices monitored by sensors within the setting 104 also appear in the legend, and their use is mapped on the half hour that ‘on’ status is detected. On this page, away-from-home time can be noted by, for example, putting a black outline around half hour intervals in which away-from-home is recorded. On the bottom left of this figure is an example of how that data can be reported on the Home Life Record® 220. On the bottom right are examples of alerts or notifications derived from this data that could be reported to the monitoring users 106.

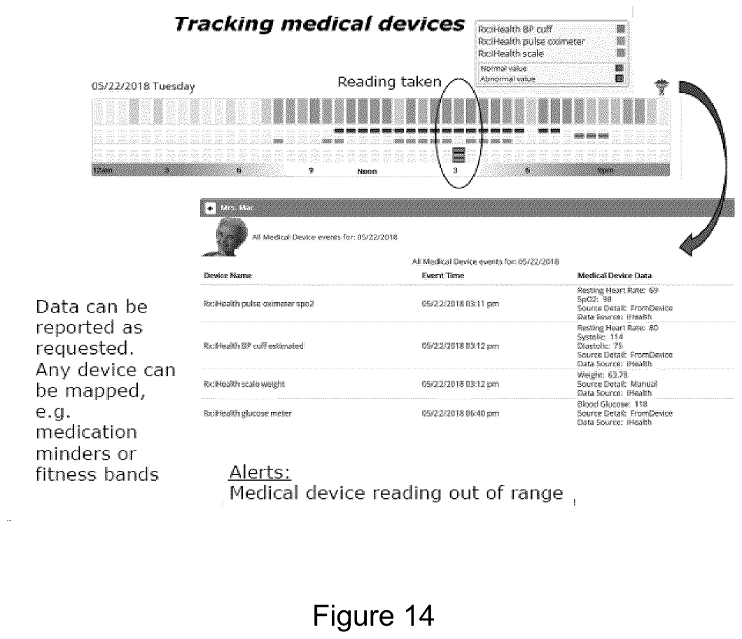

FIG. 14 is a series of related graphical illustrations usable to track medical device usage of the individual 102 (illustrated in FIG. 1) that can be provided through use of the monitoring system 100 (illustrated in FIG. 1). As described herein, these measurements can include discrete data or derived information that is presented in the symptom cluster chart related to physiological measurements either taken in the home or from other locations such as an electronic health record.

In particular, the graphical illustrations incorporate evidence and data related to medical device usage that use algorithms, statistics and trend analyses that, when viewed by the monitoring users 106 (illustrated in FIG. 1), enable the monitoring users 106 to track life events of the individual 102 visually. Such details can also be provided in any suitable manner within the Home Life Record® 220 (illustrated in FIG. 2) and/or the healthcare dashboard 980 (illustrated in FIG. 9).

As shown in FIG. 14, a monitored individual 102 could use any suitable medical devices whose output is relevant to their care. As illustrated, the Daily Activity map includes readings from three medical devices used by the monitored individual 102. Three readings were taken in the half hour interval between 3 pm and 3:30 pm. One was out of range (the blood pressure cuff reading has a red outline), and two were within the defined normal range (the pulse oximeter and weight scale). In some embodiments, by clicking on the medical icon at the top right of the day of interest, the monitoring user 106 can see the medical device data collected on that day as it comes through an API with the device manufacturer. That data can also be reported on the Home Life Record® 220, and alerts can be sent to monitoring users 106 telling them that the monitored individual has medical device readings outside of the normal range as specified.

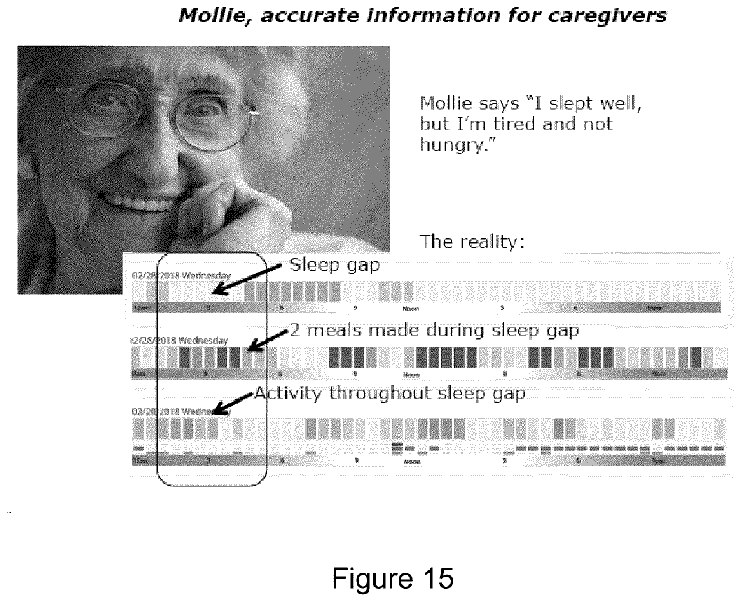

FIG. 15 is a series of related graphical illustrations usable to review sleep, meal preparation, and movement activities of the individual 102 (illustrated in FIG. 1) that can be provided through use of the monitoring system 100 (illustrated in FIG. 1), which may differ from what has been verbally reported by an individual having issues of dementia.

More specifically, FIG. 15 provides a graphical illustration focusing on a real example of a test home using the monitoring system 100 of the present invention. In this home, a family caregiver is living with an older adult with dementia (identified as “Mollie”). Persons with dementia are often unable to accurately report past events, so the dashboard can help a caregiver understand what has happened in the home when the caregiver is not present. In this particular example, this morning Mollie said she slept well but was tired and not hungry, which could be an indication she is getting sick. However, the actual data of her sleeping activities showed that in fact Mollie had been awake for much of the night, had made herself two meals, and had been pretty active. This would explain her feelings of being tired and not hungry. In reality, what Mollie needed was a nap, which is shown as occurring around noon in gray.

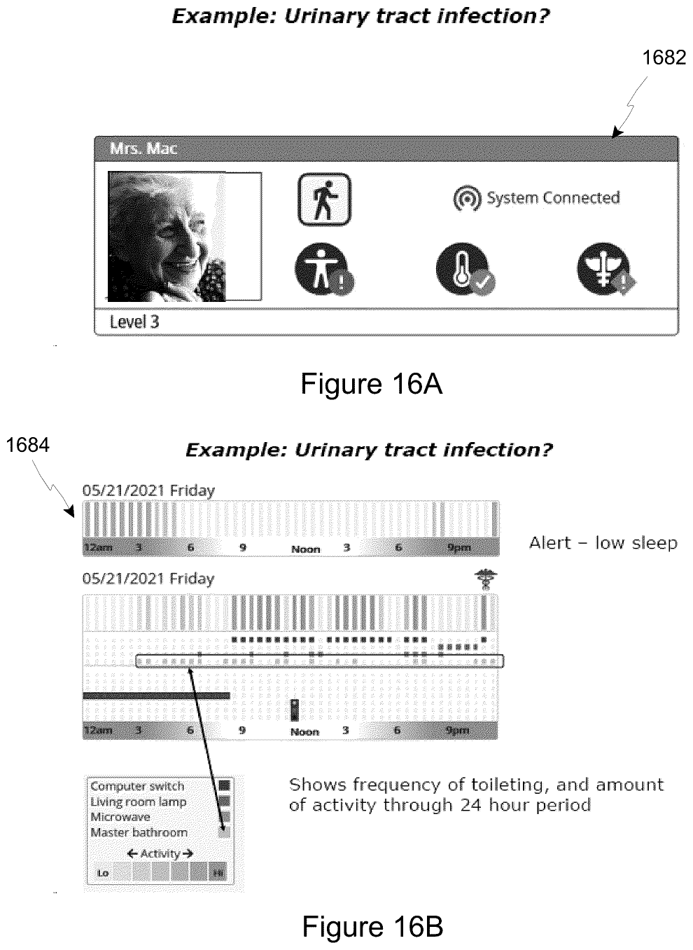

FIG. 16A is a simplified illustration of an example top level view of a general presentation 1682 regarding a potential condition of the individual 102 (illustrated in FIG. 1) that can be provided through use of the monitoring system 100 (illustrated in FIG. 1). As shown, the example presentation 1682 shows anomalies that are detected at the level of behavior and medical device readings that suggest a need to examine a more detailed view of the individual, including making real time contact.

More particularly, the general presentation 1682 for the individual 102 (identified as “Mrs. Mac”) shows an orange banner, indicating that an anomaly or abnormal reading has been recorded. Along the bottom of the general evaluation card, the data indicator for behavior shows a red exclamation point, indicating to the monitoring users 106 that a potentially significant change has been detected in data related to daily activity. As further shown, a medical device reading is also outside the defined normal range. Based on such sensed data, it is determined that further analysis regarding consistency with a potential condition, such as urinary tract infection, would be prudent.

FIG. 16B is a simplified illustration of an example detailed view of certain factors for the general presentation 1684 regarding the potential condition of the individual of FIG. 16A that can be provided through use of the monitoring system 100 (illustrated in FIG. 1). As shown in this specific example, the individual (“Mrs. Mac”) is shown to have been asleep at midnight, but was then awake after approximately 4 am. As interpreted, there was quiet in the bedroom, but it did not meet the inference engine criteria of sleep (3 gray columns). At the same time, the daily activity map shows low levels of activity starting around 3 am. In particular, a sensor in the master bathroom shows repeated activity from 3 am onward throughout the day. Based on this sensed data, an alert for low sleep was generated and sent to the monitoring users 106.

It is understood, however, that the data is not actually intended to diagnose or treat conditions; however, changes that could be meaningful relative to a particular condition can be reported to the monitoring users 106 so the monitored individual 102 gets attention when it is likely to help that monitored individual 102 remain safer and healthier at home. In this case, the sensed data indicates that it would be appropriate to test for a potential urinary tract infection, with early detection and treatment being highly beneficial for the monitored individual 102.

In summary, as described in detail herein, the various Figures describe the monitoring system 100, how it is used, and how the generated data would feed into a flexible graphic, i.e. the visualized compilation data set such as a symptom cluster chart, that contains both summary and detailed information related to a descriptor with multiple inputs. For example, as noted, the descriptor “sleep” could include timing, duration, and consistency measurements. Further, consistency could include measuring gaps each night and comparing the measurement to other nights to determine whether those gaps are part of a normal pattern or an anomaly for the night in question.

Also, a disease episode can have multiple indicators that are different from the personal normal baseline of the care recipient. For example, a person experiencing a congestive heart failure (CHF) episode can gain water weight, have increasing difficulty sleeping, lose appetite, and have less energy. The graphic would provide information at a high level for each of those symptoms, and then each symptom would be divided into its own inputs for measurements, and each of those inputs further measured until the graphic reflects the primary data (from a sensor or other data source).

It is understood that although a number of different embodiments of the monitoring system have been illustrated and described herein, one or more features of any one embodiment can be combined with one or more features of one or more of the other embodiments, provided that such combination satisfies the intent of the present invention.

While a number of exemplary aspects and embodiments of monitoring system have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope, and no limitations are intended to the details of construction or design herein shown.

What is claimed is:

1. A system for generating a symptom cluster chart relevant to at least one condition of an individual in a setting, the system comprising:
a data analytics resource that is configured to:
generate three or more input axes for the symptom cluster chart that extend outwardly from a common origin, each of the three or more input axes relating to a condition parameter of the at least one condition;
receive data from a plurality of data sources for each condition parameter relative to a predetermined period of time;
determine a normal range for each condition parameter based on the received data;
assign numeric value along each of the three or more input axes for each condition parameter so that the normal range for each condition parameter forms a first regular polygon shape when plotted and connected along each of the input axes on the symptom cluster chart;
establish a threshold of acceptable deviation from the normal range for each condition parameter so that the threshold of acceptable deviation for each condition parameter forms a second regular polygon shape when plotted and connected along each of the input axes on the symptom cluster chart;
receive at least one additional data point for each condition parameter;
plot the at least one additional data point for each condition parameter along the three or more input axes on the symptom cluster chart;
connect the at least one additional data point for each condition parameter on the symptom cluster chart to form an updated polygon; and
graphically illustrate the symptom cluster chart on a graphical user interface.

2. The system of claim 1 wherein the second regular polygon shape is larger than the first regular polygon shape.

3. The system of claim 1 wherein at least one of the plurality of data sources is positioned within the setting.

4. The system of claim 1 wherein at least one of the plurality of data sources is positioned outside the setting.

5. The system of claim 1 wherein the at least three input axes are weighted relative to one another within the symptom cluster chart.

6. The system of claim 1 wherein if the at least one additional data point from any of the plurality of data sources is outside the threshold of acceptable deviation for the condition parameter, then the symptom cluster chart graphically illustrated on the graphical user interface changes in one or more of shape, size, fill pattern, and color.

7. The system of claim 6 wherein if each additional data point from the plurality of data sources is outside the threshold of acceptable deviation for the condition parameter, then the symptom cluster chart graphically illustrated on the graphical user interface changes in both size and color.

8. The system of claim 1 wherein the condition parameters relevant to the at least one condition of the individual include one or more of sleep activities, eating activities, movement activities, environmental readings, and medical device readings relative to the individual.

9. The system of claim 8 wherein the condition parameters relevant to the at least one condition of the individual include the sleep activities of the individual; and
wherein the sleep activities of the individual include sleep time, wake time, sleep duration, and sleep gaps as sensed or inferred by the plurality of data sources.

10. The system of claim 1 wherein the at least one condition of the individual includes frailty; and wherein the condition parameters include (i) eating/weight, (ii) amount/distribution of activity, (iii) walking speed, (iv) fatigue, and (v) socialization.

11. The system of claim 1 wherein the at least one condition of the individual includes congestive heart failure; and wherein the condition parameters include (i) pillow count, (ii) blood pressure, (iii) activity at night, (iv) fatigue, (v) amount of movement during day, (vi) weight, (vii) sleep gaps, and (viii) oxygen saturation.

12. The system of claim 1 further comprising a data collection resource that is configured to collect and store the data from the plurality of data sources for each of the condition parameters.

13. The system of claim 12 wherein the data collection resource is a cloud-based system.

14. The system of claim 13 wherein the data analytics resource is incorporated within the data collection resource.

15. A method for generating a symptom cluster chart relevant to at least one condition of an individual in a setting, the method comprising steps of:
generating with a data analytics resource three or more input axes for the symptom cluster chart that extend outwardly from a common origin, each of the three or more input axes relating to a condition parameter of the at least one condition;
receiving data from a plurality of data sources for each condition parameter relative to a predetermined period of time with the data analytics resource;
determining a normal range for each condition parameter based on the received data with the data analytics resource;
assigning numeric value along each of the three or more input axes for each condition parameter with the data analytics resource so that the normal range for each condition parameter forms a first regular polygon shape when plotted and connected along each of the input axes on the symptom cluster chart;
establishing a threshold of acceptable deviation from the normal range for each condition parameter with the data analytics resource so that the threshold of acceptable deviation for each condition parameter forms a second regular polygon shape when plotted and connected along each of the input axes on the symptom cluster chart;
receiving at least one additional data point for each condition parameter with the data analytics resource;
plotting the at least one additional data point for each condition parameter along the three or more input axes on the symptom cluster chart with the data analytics resource;
connecting the at least one additional data point for each condition parameter on the symptom cluster chart with the data analytics resource to form an updated polygon; and
graphically illustrating the symptom cluster chart on a graphical user interface with the data analytics resource.

16. The method of claim 15 wherein the step of receiving includes at least one of the plurality of data sources being positioned within the setting.

17. The method of claim 15 wherein the step of receiving includes at least one of the plurality of data sources being positioned outside the setting.

18. The method of claim 15 wherein the step of graphically illustrating includes if the at least one additional data point from any of the plurality of data sources is outside the threshold of acceptable deviation for the condition parameter, then the symptom cluster chart graphically illustrated on the graphical user interface changes in one or more of shape, size, fill pattern, and color.

19. The method of claim 15 further comprising a step of collecting and storing the data from the plurality of data sources for each of the condition parameters with a data collection resource.

20. The method of claim 19 wherein the step of collecting and storing includes the data collection resource being a cloud-based system; and wherein the data analytics resource is incorporated within the data collection resource.
